(19) United States
(12) Patent Application Publication
(10) Pub. No.: US 2025/0122228 A1
(43) Pub. Date: Apr. 17, 2025

(54) METAL-ORGANIC COORDINATION COMPOUND COMPRISING CERIUM(III)

(71) Applicant: beeOLED GmbH, Dresden (DE)

(72) Inventors: Carsten ROTHE, Dresden (DE); Volodymyr SENKOVSKYY, Dresden (DE)

(73) Assignee: beeOLED GmbH, Dresden (DE)

(21) Appl. No.: 18/907,407

(22) Filed: Oct. 4, 2024

Related U.S. Application Data
(63) Continuation of application No. PCT/EP2023/058811, filed on Apr. 4, 2023.

(30) Foreign Application Priority Data
Apr. 4, 2022 (EP) 22166525.0

(51) Int. Cl. C07F 7/00; H10K 71/16; H10K 85/30; H10K 85/60

(52) U.S. Cl. CPC C07F 7/00; H10K 71/164; H10K 85/351; H10K 85/60

(57) ABSTRACT

A metal-organic coordination compound, wherein the coordination compound comprises at least one Cerium (III) coordinated by a poly macrocyclic organic ligand having the formula 1-1

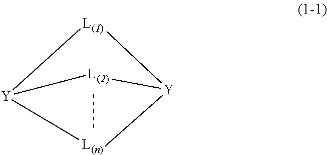

wherein n is an integer of at least 3,
Y at each occurrence is independently a n-valent group containing 1 to 30 atoms,
L at each of its n occurrences is independently a divalent organic group, wherein L has a sequence of at least 3 atoms, preferably at least 5 atoms connecting both Y in case that Y is a cyclic group connected with each L via different ring atoms of Y,
wherein L has a sequence of at least 5 atoms connecting both Y in case that Y is connected with each L via the same atom of Y,
wherein the poly macrocyclic organic ligand comprises at least three nitrogen atoms.

This nonprovisional application is a continuation of International Application No. PCT/EP2023/058811, which was filed on Apr. 4, 2023, and which claims priority to European Patent Application No. 22166525.0, which was on Apr. 4, 2022, and which are both herein incorporated by reference.

### BACKGROUND OF THE INVENTION

### Field of the Invention

The invention relates to a metal-organic coordination compound, wherein the coordination compound comprises at least one Cerium (III) coordinated by a polymacrocyclic organic ligand, a mixture containing the metal-organic coordination compound, a polymeric compound, wherein the coordination compound is covalently attached to the polymer backbone, an organic electronic device containing them, methods for forming the same as well as the use of the coordination compound.

### Description of the Background Art

Electroluminescent devices that make use of organic light emitting diodes (OLEDs) are state-of-the-art for flat panel display applications used in everyday consumer electronics. For OLEDs usually special organic materials are employed for the purpose of converting electrical excitation into light emission. For most organic emitters, the excitation that is formed upon recombination of an electron and a hole on such an emitter molecule is called an “exciton”. Depending on the spins of the recombining charges, there are two types of excitons formed in a statistical manner: 75% probability of triplet excitons with spin 1, and 25% probability singlet excitons with spin zero are generated. If the emitter molecule is heteroaryl-based without any significant content of heavy metals, then, because of spin-conservation, only the singlet excitons contribute to light emission, known as fluorescence (FL). Thus, fluorescent OLEDs are comparably inefficient as 75% of the invested electrical power is wasted.

In the conventional art, incorporation of quantum mechanical heavy metal effect into the emitter molecules, by introducing d-metal elements such as Iridium, Osmium, Gold or Platinum, has been used. The presence of heavy metal elements softens the selection rules for the excited states and allows triplet excitons to emit light too; known as phosphorescence (Ph).

Thermally activated delayed fluorescence (TADF) has been used wherein by thoughtful design of the organic emitter molecule, the energy difference between the non-emissive triplet and the emissive singlet exciton is engineered to be very small. This allows triplets to thermally convert into singlet excitons and thereby contribute to light emission.

However, there are no TADF or Ph blue emitters with sufficient chemical stability known which hinders their implementation into OLED applications. The underlying cause of low chemical stability is due to the formation of charge-separated states upon excitation. For Ph emitters, the exciton resides on two different parts of the emitter, namely the central heavy metal cation and the organic ligand. During excitation, especially with high energetic blue light, the chemical bond between metal center and organic ligand is weakened, giving rise to chemical decomposition. Similarly, for TADF emitters, a low energy difference between singlet and triplet exciton energy is needed, which is achieved by bridging the excitation in-between an electron accepting and an electron donating part. Again, upon excitation with energies corresponding to blue photons, the chemical organic bond between those two parts of the emitter molecules are substantially weakened, giving rise to bond cleavage and in consequence short operational lifetime in OLED device.

Therefore, TADF or Ph blue emitting materials cannot be used in display applications, as otherwise the blue spectral component would fade away after prolonged operation times which is known as burn-in.

Thus, for emitter molecules suitable for OLED, it is desirable to avoid charge separated states, but to localize the excitation on one part of the molecule, preferably on a single atom. Generally, elements with suitable intra-atomic transitions are found within the f-Elements, i.e. the lanthanides. Emitters based on such lanthanides have been extensively used in OLEDs. For example, OLEDs based on intra-atomic f-f transitions in the blue, green, and red spectral region based on Thulium (Tm3+), Terbium (Ter3+) and Europium (Eu3+) respectively have been demonstrated. However, OLED emitters based on the above f-f transitions have a serious flaw which renders them as being unsuitable for display applications. Here, the excited state relaxation time is around one millisecond, which is about three orders of magnitude too long for display applications. Such slow relaxation times are incompatible with the requirements of fast display content refresh rate and as well lead to a severe efficiency roll-off of the OLEDs at high brightness. Here, a long-excited state lifetime leads to a high density of excitations in the active OLED layer, which leads to bimolecular annihilation loss and low efficiency.

Fast excited state lifetimes for lanthanides are possible if the lowest optical transition is of the Laporte allowed d-f type. Here Europium2+ shows lifetimes of around 1 us and especially Cerium3+ features lifetimes of about 50-100 ns.

The preferred oxidation state of all lanthanide metals is trivalent. Thus, a major obstacle for the application of divalent Europium is its chemical instability, and—in particular—its tendency to oxidize under normal ambient conditions, which renders its application to OLED extremely difficult.

The natural lowest allowed optical transition of Ce3+ occurs in the deep UV spectral region invisible for the human eye and thus not useful for OLED application. However, complexing the metal with soft, organic ligands its emission can be shifted into the blue spectral region as desirable for OLED application. Indeed complexing Ce3+ with two tripodal pyrazolyl ligands deep blue, efficient OLEDs have been demonstrated, as described by: Zheng et al., Angew. Chem. Int. Ed 2007, 46, 7399-7403; Wang et al., Light Science and Applications (2020) 9:157; and Fang et al., ACS Appl. Mater. Interfaces 2021, 13, 45686-45695.

A serious drawback of employing multiple flexible organic ligands is the unavoidable large excited state relaxation upon excitation of the metal organic complex, which is usually referred to as Stokes shift. Such flexible environments of the metal open pathways for non-radiative relaxation and for undesired chemical reactions leading to degradation. Indeed disclosed operational lifetimes of Cerium-pyrazolyl type emitter are at the orders of minutes only, preventing there application in consumer electronics.

Desirable non-flexible and rigid organic environments for metals can be provided by complexing the metal using a single macrocyclic or polymacrocyclic multidentate ligand such as crownethers or cryptands. In related arts for OLED applications, porphyrins are known as red emitters, specific cryptands have been proposed as ligands for d-metal Ph emitters, crown ether or cryptands have been proposed to stabilize reactive metals for n-type doping, and cryptands are demonstrated to transport ions in light emitting cells.

Metal organic Cerium3+ complexes comprising macrocyclic ligands are readily known. For example Frey et.al. and Brucher et.al. study crownether based Ce3+ complexes whereas Blasse et.al. discloses cryptands with Ce3+. In all those cases the central Cerium is coordinated by hard oxygens implying that the emission is in the UV, unsuitable for OLED applications.

In a related art, Yu et.al. attempts to provide a UV-emitting electroluminescent device using a Cerium3+ metal-organic complex based on dibenzocrown ether ligand. The OLED is supposed to be manufactured by thermal vacuum processing of the Cerium based emitter material. However, a closer inspection by us reveals that upon heating in high vacuum the emitter material completely decomposes into a volatile ligand and non-volatile Ceriumchlorid. Consistent with this finding, the electroluminescent UV light observed by Yu et.al. can by solely explained by carbazole based host emission.

Cerium3+ coordination compounds feature attractive emission characteristics due to their Laporte allowed d-f intra-atomic transition with their short excited state lifetimes. Applied in a rigid organic environment those cations may exhibit sufficient chemical stability preventing them from fast electro-chemical degradation thereby making them applicable for a large range of opto-electronic devices, for example sensors, solar cells, electroluminescent devices, or color conversion materials. A suitable rigid organic environment is provided using multidentate polymacrocyclic organic ligands. However, at the same time the latter ligands have to be electronically soft enough to shift the originally UV Cerium optical transition into the desirable blue spectral region. Thus, so far no Cerium coordination compounds are known that do provide the rigid organic environment needed for commercially attractive organic electronic application combined with the softness to give attractive deep blue color.

### SUMMARY OF THE INVENTION

The object is achieved by a metal-organic coordination compound, wherein the coordination compound comprises at least one Cerium (III) coordinated by a polymacrocyclic organic ligand having the formula 1-1

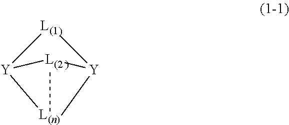

wherein n is an integer of at least 3,
Y at each occurrence is independently a n-valent group containing 1 to 30 atoms,
L at each of its n occurrences is independently a divalent organic group, wherein L has a sequence of at least 3 atoms, preferably at least 5 atoms connecting both Y (i.e. between the two linkages with Y) in case that Y is a cyclic group connected with each L via different ring atoms of Y,
wherein L has a sequence of at least 5 atoms connecting both Y (i.e. between the two linkages with Y) in case that Y is connected with each L via the same atom of Y,
   wherein the polymacrocyclic organic ligand comprises at least three nitrogen atoms.

In various aspects of the invention an electroluminescent coordination compound, a mixture, a polymeric compound and an organic electronic device as well as methods for forming the same are provided.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various aspects of the invention are described with reference to the following drawings, in which:

FIGS. 1 and 10 illustrate polymacrocyclic organic ligands according to various examples;

FIGS. 2, 3, and 5 illustrate formulas of various mono- and divalent side groups of polymacrocyclic organic ligands;

FIG. 4 illustrates negatively charged anions according to various examples;

FIG. 6a and FIG. 6b illustrate repeat units of polymeric compounds according to various examples;

FIG. 7 illustrate formulas of various coordination compounds according to examples of this invention;

FIG. 8A and FIG. 8B illustrate schematic cross sections of organic electronic devices according to various aspects;

FIG. 9 illustrates a flow diagram of a method for producing a coordination compound according to various aspects;

FIG. 11 depicts a chemical reaction scheme for the production of a particular coordination compound;

FIG. 12 shows emission spectra of various coordination compounds; and

FIG. 13 shows characteristics of an OLED device comprising a particular coordination according to this invention.

### DETAILED DESCRIPTION

The following detailed description refers to the accompanying drawings that show, by way of illustration, specific details and aspects in which the invention may be practiced.

The word “exemplary” is used herein to mean “serving as an example, instance, or illustration”. Any aspect or design described herein as “exemplary” is not necessarily to be construed as preferred or advantageous over other aspects or designs.

Various examples relate to metal organic compounds, comprising Ce (III) coordinated with a poly macrocyclic organic ligand including a plurality of aliphatic amine groups, the production of such compounds and their applications.

In this description, a coordination compound is taken to mean a compound where the central active metal cation is coordinated without a direct metal carbon bond.

In this description, an electroluminescent coordination compound is any material that is able to emit light upon electrical excitation, followed by recombination of electrons and holes. It shall be irrelevant in this context, whether the recombination of the electrons and holes takes place directly on the electroluminescent compound or first an excitation is formed on a different compound and subsequently transferred to the electroluminescent compound. Further, the electroluminescent coordination compound does not necessarily have to be used in an electronic device but, as example, may be used as a dye or color conversion material.

In this description, the arylene is a divalent fragment that is derived from an aromatic or heteroaromatic hydrocarbon by removing two hydrogen atoms from the aromatic or heteroaromatic hydrocarbon, preferably from different carbon and/or hetero atoms. One example is a (hetero) aromatic hydrocarbon that has had hydrogen atoms removed from two, preferably adjacent, hydrogen-bearing atoms (in case of aromatic hydrocarbon two carbon atoms, in case of heteroaromatic hydrocarbons two atoms selected from carbon and heteroatoms). An aromatic hydrocarbon or arene (or sometimes aryl hydrocarbon) is a hydrocarbon with sigma bonds and delocalized pi electrons between carbon atoms forming a circle.

The terms “halo,” “halogen,” and “halide” are used interchangeably and refer to fluorine, chlorine, bromine, and iodine.

The term “acyl” refers to a substituted carbonyl radical (C(O)—Rs).

The term “ester” refers to a substituted oxycarbonyl (—O—C(O)—Rs or —C(O)—O—Rs) radical.

The term “ether” refers to an —ORs radical.

The terms “sulfanyl” or “thio-ether” are used interchangeably and refer to a —SRs radical.

The term “sulfinyl” refers to a —S(O)—Rs radical.

The term “sulfonyl” refers to a —SO2—Rs radical.

The term “phosphino” refers to a —P(Rs)3 radical, wherein each Rs can be same or different.

The term “silyl” refers to a —Si(Rs)3 radical, wherein each Rs can be same or different.

In each of the above, Rs can be hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combination thereof. Preferred Rs are selected from the group consisting of alkyl, cycloalkyl, aryl, heteroaryl, and combination thereof.

The term “alkyl” refers to and includes both straight and branched chain alkyl radicals. Preferred alkyl groups are those containing from one to fifteen carbon atoms and include methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. The alkyl group may be optionally substituted, preferably with halogen, in particular with F.

The term “cycloalkyl” refers to and includes monocyclic, polycyclic, and spiro alkyl radicals. Preferred cycloalkyl groups are those containing 3 to 12 ring carbon atoms and includes cyclopropyl, cyclopentyl, cyclohexyl, bicyclo[3.1.1] heptyl, spiro [4.5] decyl, spiro [5.5] undecyl, adamantyl, and the like. Additionally, the cycloalkyl group is optionally substituted.

The terms “heteroalkyl” or “heterocycloalkyl” refer to an alkyl or a cycloalkyl radical, respectively, having at least one carbon atom replaced by a heteroatom. Optionally the at least one heteroatom is selected from O, S, N, P, B, Si and Se, preferably O, S or N. Additionally, the heteroalkyl or heterocycloalkyl group is optionally substituted.

The term “alkenyl” refers to and includes both straight and branched chain alkene radicals. Alkenyl groups are essentially alkyl groups that include at least one carbon-carbon double bond in the alkyl chain. Cycloalkenyl groups are essentially cycloalkyl groups that include at least one carbon-carbon double bond in the cycloalkyl ring. The term “heteroalkenyl” as used herein refers to an alkenyl radical having at least one carbon atom replaced by a heteroatom. Optionally the at least one heteroatom is selected from O, S, N, P, B, Si, and Se, preferably O, S, or N. Preferred alkenyl, cycloalkenyl, or heteroalkenyl groups are those containing two to fifteen carbon atoms. Additionally, the alkenyl, cycloalkenyl, or heteroalkenyl group is optionally substituted.

The terms “aralkyl” or “arylalkyl” are used interchangeably and refer to an alkyl group that is substituted with an aryl group. Additionally, the aralkyl group is optionally substituted.

The term “heterocyclic group” refers to and includes aromatic and non-aromatic cyclic radicals containing at least one, preferably 1 to 5, more preferably 1 to 3, most preferably 1 or 2 heteroatom. Preferably the at least one heteroatom is selected from O, S, N, P, B, Si, and Se, more preferably O, S, or N. Hetero-aromatic cyclic radicals may be used interchangeably with heteroaryl. Preferred hetero-non-aromatic cyclic groups are those containing 3 to 7 ring atoms which includes at least one hetero atom, and includes cyclic amines such as morpholino, piperidino, pyrrolidino, and the like, and cyclic ethers/thio-ethers, such as tetrahydrofuran, tetrahydropyran, tetrahydrothiophene, and the like. Additionally, the heterocyclic group may be optionally substituted, e.g. by halogen, alkyl or aryl. The heterocyclic group can be covalently linked with the remainder of the molecule via carbon and/or heteroatoms, preferably one carbon or nitrogen atom.

The term “aryl” refers to and includes both single-ring aromatic hydrocarbyl groups and polycyclic aromatic ring systems. The polycyclic rings may have two or more rings in which two carbons are common to two adjoining rings (the rings are “fused”) or wherein one carbon is common to two adjoining rings (e.g.biphenyl) wherein at least one of the rings is an aromatic hydrocarbyl group, e.g., the other rings can be cycloalkyls, cycloalkenyls, aryl, heterocycles, and/or heteroaryls. Preferred aryl groups are those containing six to thirty carbon atoms, preferably six to twenty carbon atoms, more preferably six to twelve carbon atoms. Especially preferred is an aryl group having six carbons, ten carbons or twelve carbons. Suitable aryl groups include phenyl, biphenyl, triphenyl, triphenylene, tetraphenylene, naphthalene, anthracene, phenalene, phenanthrene, fluorene, pyrene, chrysene, perylene, radialene and azulene, preferably phenyl, and pyrene. Additionally, the aryl group is optionally substituted, e.g. by halogen, alkyl, heteroalkyl.

In many instances individual aryl groups containing at most eight aromatic carbon atoms are preferred, whereby various aryl groups maybe present as long as the aromatic carbons are separated from each other by at least one aliphatic carbon or heteroatom.

The term “heteroaryl” refers to and includes both single-ring aromatic groups and polycyclic aromatic ring systems that include at least one heteroatom. The heteroatoms include, but are not limited to and are preferably selected from O, S, N, P, B, Si, and Se. In many instances, O or N are the preferred heteroatoms. Hetero-single ring aromatic systems are preferably single rings with 5 or 6 ring atoms, and the ring can have from one to five/six, preferably 1 to 3, more preferably 1 or 2 heteroatoms. The hetero-polycyclic ring systems can have two or more rings in which two atoms are common to two adjoining rings (the rings are “fused”) or wherein one carbon is common to two adjoining rings (e.g. bipyridine) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to five/six, preferably 1 to 3, more preferably 1 or 2 heteroatoms per ring of the polycyclic aromatic ring system. Preferred heteroaryl groups are those containing three to thirty carbon atoms, preferably three to twenty carbon atoms, more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuran, benzofuropyridine, furodipyridine, benzothienopyridine, thienodipyridine, benzoselenophenopyridine, 1,2-azaborine, 1,3-azaborine, 1,4-azaborine, borazine, and aza-analogs thereof, from which one hydrogen atom has been removed from a hydrogen-bearing carbon or heteroatom to form the covalent link to the remainder of the molecule, and selenophenodipyridine, preferably dibenzofuran, benzofuran, furan, dioxazole, carbazole, pyridine, pyridazine, pyrimidine, and pyrazine. Additionally, the heteroaryl group is optionally substituted, e.g. by halogen, alkyl or aryl.

Of the aryl and heteroaryl groups listed above, the groups of benzene, furan, dibenzofuran, carbazole, and pyridine are of particular interest.

In many instances individual heteroaryl groups containing at most eight aromatic carbon atoms are preferred, whereby various heteroaryl or aryl groups maybe present as long as the aromatic moities are separated from each other by at least one aliphatic carbon or heteroatom.

The alkyl, cycloalkyl, heteroalkyl, heterocycloalkyl, alkenyl, cycloalkenyl, heteroalkenyl, alkynyl, aralkyl, heterocyclic group, aryl, and heteroaryl groups or residues, as used herein, are independently unsubstituted, or independently substituted, with one or more (general) substituents, preferably the substituents mentioned above.

Preferably, the (general) substituents are selected from the group consisting of deuterium, halogen, CN, NO₂, haloalkyl, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof with the number of carbon atoms and heteroatoms as defined above for the respective term. Furthermore, one or two substituents can be selected from polymer chains which can be covalently linked with the remainder of the molecule by a suitable organic spacer. Therefore, the cyclic organic ligand can be covalently linked with a polymer chain or a polymer backbone, or a suitably modified surface.

In some instances, the preferred general substituents are selected from the group consisting of deuterium, carbazole, dibenzofuran, alkyl, cycloalkyl, heteroalkyl, alkoxy, aryloxy, amino, silyl, alkenyl, cycloalkenyl, heteroalkenyl, aryl, heteroaryl, nitrile, isonitrile, sulfanyl, and combinations thereof with the number of carbon atoms and heteroatoms as defined above for the respective term.

In some instances, the preferred general substituents are selected from the group consisting of deuterium, carbazole, dibenzofuran, alkyl, cycloalkyl, alkoxy, aryloxy, amino, silyl, aryl, heteroaryl, sulfanyl, and combinations thereof.

In yet other instances, the more preferred general substituents are selected from the group consisting of carbazole, dibenzofurane, alkyl, cycloalkyl, aryl, heteroaryl, and combinations thereof.

The terms “substituted” and “substitution” refer to a substituent other than H that is bonded to the relevant position, e.g., a carbon or nitrogen. For example, when R1 represents mono-substitution, then one R1 must be other than H (i.e., a substitution). Similarly, when R1 represents di-substitution, then two of R1 must be other than H. Similarly, when R1 represents no substitution, R1, for example, can be a hydrogen for available valencies of straight or branched chain or ring atoms, as in carbon atoms for benzene and the nitrogen atom in pyrrole, or simply represents nothing for ring atoms with fully filled valencies, e.g., the nitrogen atom in pyridine. The maximum number of substitutions possible in a straight or branched chain or ring structure will depend on the total number of available valencies in the ring atoms or number of hydrogen atoms that can be replaced. All residues and substituents are selected in a way that a chemically stable and accessible chemical group results.

As used herein, “combinations thereof” indicates that one or more members of the applicable list are combined to form a known or chemically stable arrangement that one of ordinary skill in the art can envision from the applicable list. For example, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium counted for all substituents of a given molecule, or for the respective molecule in total. In many instances, a preferred combination of substituent groups will include up to twenty atoms that are not hydrogen or deuterium, counted for all substituents of a given molecule.

The “aza” designation in the fragments described herein, i.e. aza-cryptate, etc. means that one or more of the C—H groups in the respective aromatic ring is replaced by a nitrogen atom, and without any limitation. One of ordinary skill in the art can readily envision other nitrogen analogs of the aza-derivatives, and all such analogs are intended to be encompassed by the terms as set forth herein.

As used herein, “deuterium” refers to an isotope of hydrogen. Deuterated compounds can be readily prepared using methods known in the art.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

In some instances, a pair of adjacent or non-adjacent substituents or residues can be optionally joined (i.e. covalently linked with each other) or fused into a ring. The preferred ring formed therewith is a five-, six-, or seven-membered carbocyclic or heterocyclic ring, including both instances where the portion of the ring formed by the pair of substituents is saturated and where the portion of the ring formed by the pair of substituents is unsaturated. As used herein, “adjacent” means that the two substituents involved can be on the same ring next to each other, or on two neighboring rings having the two closest available substitutable positions, such as 2,2′ positions in a biphenyl, or 1,8 position in a naphthalene, or 2,3-positions in a phenyl, or 1,2-positions in a piperidine, as long as they can form a stable fused ring system.

The term macrocyclic refers to cyclic organic molecules comprising at least 9 atoms of which at least one is a heteroatom according to definition above.

A polymacrocyclic organic comprises at least two macrocylic rings that are covalently linked to each other. Irrelevant in this context is whether two such macrocylic rings are linked to each other just ones or multiple times.

A bis macrocyclic organic refers to a molecule containing exactly two macrocyclic rings being once or multiple times covalently linked to each other. A bis macrocyclic is part of the family of poly macrocyclic organic ligands.

The Cerium (III) coordination compound according to invention comprises a polymacrocyclic organic ligand, which, as a consequence of its many coordination sites and 3D structure, provides a rigid enviroment of the central electro-active cation thereby reducing undesired conformational relaxation. Simultaneiously, this polymacrocyclic ligand shields the central cation and the emission light emission process from the enviroment such as sourrounding host materials and thereby ensures high light emission efficiencies and inertness against chemical degradation.

The polymacrocyclic organic ligand is described by formula 1-1:

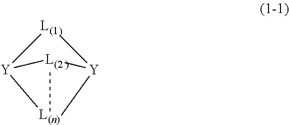

wherein n is an integer of at least 3,
Y at each occurrence is independently a n-valent group containing 1 to 30 atoms,
L at each of its n occurrences is independently a divalent organic group, wherein L has a shortest sequence of at least 3 atoms, preferably at least 5 atoms directly connecting both Y (i.e. between the two linkages with Y) in case that Y is a cyclic group connected with each L via different ring atoms of Y,
wherein L has a shortest sequence of at least 5 atoms directly connecting both Y (i.e. between the two linkages with Y) in case that Y is connected with each L via the same atom of Y,
   wherein the polymacrocyclic organic ligand comprises at least three nitrogen atoms. The macrocycles of the macrocyclic organic ligand of formula (1-1) are the cycles formed by each pair of L and the Y groups. Y itself can be macrocyclic, e.g. a cyclic group containing 9 or 12 ring atoms.

Formula 1-1 describes a chemical structure having a polymacrocyclic ligand. Individual macrocyclic substructures are formed if “Y” itself is a macrocyclic group. Similar each two elements of “L” linked via “Y” do form a macrocyclic ring. For n=3 and Y being a nitrogen atom the structure thus describes a biscyclic organic ligand, known as cryptand. The linker “L” as well as “Y” in case it is a cyclic group may be composed of sub-sequences such as S—C—C or N—C—C or O—C—C or S—C—C—C or N—C—C—C or O—C—C—C or S—C or N—C or O—C, preferably S—C—C or N—C—C or O—C—C. Each of these sub-sequences may be substituted or non-substituted. In case two sub-sequences are substituted, two substitutes may link to each other, thereby forming a cyclic ring. The number, n, of independently selected linkage units “L” is greater than 2. In case “Y” is in both occurrences a cyclic or macrocyclic group, n is preferred to be 3 or 4. In case various Ls are linked via a single atom, n is preferred to be 3, thus resulting in a bis cyclic organic ligand.

Preferably, each Y is independently selected from B, B—R—, N, P, C—R, more preferably from N and C—R, most preferably from N; or each Y is independently selected from 3- to 15-membered, preferably 9- to 15 membered macrocyclic organic groups that can contain one to four heteroatoms selected from N, B, P, O, S, and is connected with each L via non-neighboring carbon or hetero atoms, wherein R is hydrogen or any covalently bound substituent, as indicated below, being identical or different at each occurrence. Preferred heteroatoms are S and N and O, the most preferred heteroatom is N.

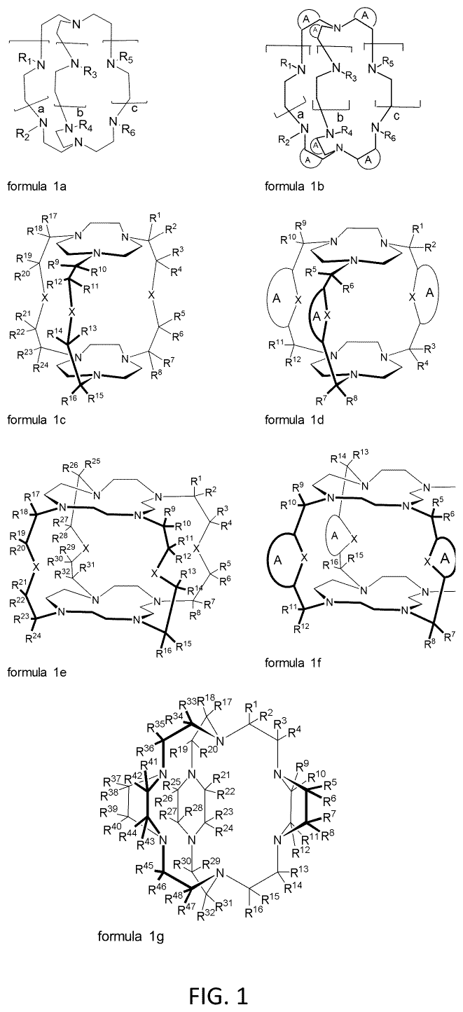

In various examples, the poly macrocyclic organic ligand according to formula 1-1 may be constructed using instances of —X—C—C— with X being a heteroatom selected from selected from O, S, N, P, B, Si, and Se. Preferred heteroatoms X are S and N and O, most preferred is N. Illustrative generic chemical formulas 1a-1f of such poly macrocyclic organic ligands are depicted in FIG. 1. Wherein
R1, -R48, in any of the generic formulas shown in FIG. 1 independently in each occurrence represent hydrogen, or an organic substituents,
a, b, c in formula 1a and 1b are each independently an integer of 0 or more,

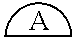

independently in each occurrence represents a divalent cyclic organic group, and
X independently in each occurrence represents a hetero atom selected from O, S, N, P, B, Si, and Se.

In FIG. 1a, b, g “Y” of formula 1-1 is formed by a macrocyclic crownether, whereas in FIG. 1c, d, e, f “Y” is made by a single nitrogen atom. In many instances, S or O or N are the preferred heteroatoms represented by “X”. This way the formulas illustrated in FIG. 1 all contain a plurality of the relatively soft coordinating nitrogen atom.

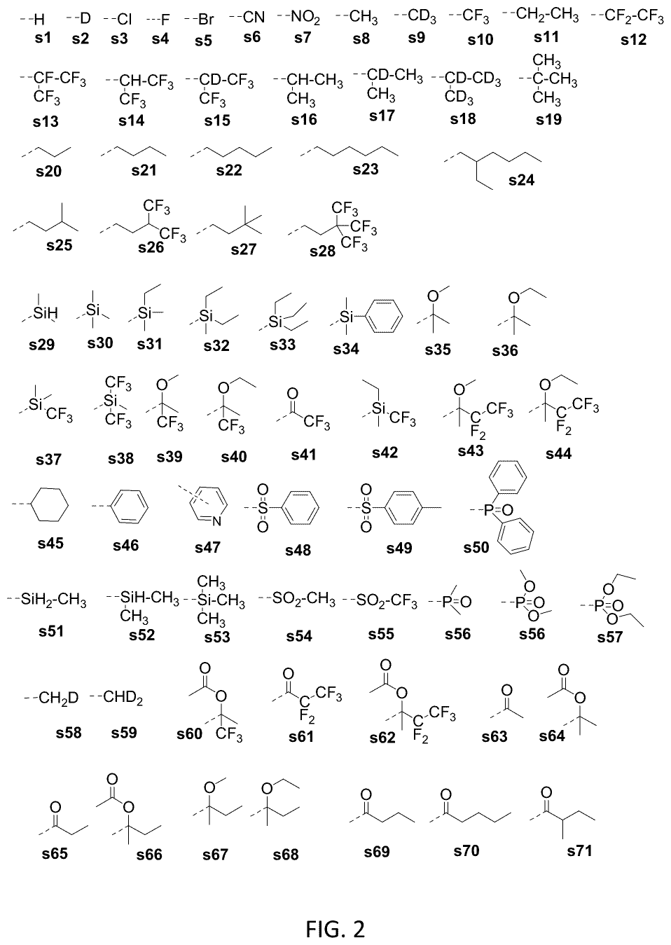

Every carbon or heteroatom with valence greater 2 present in L or Y in formula 1-1 may have side groups. R1-R48 represent such side groups in formula 1a-1g in FIG. 1. Those substituents may be any chemical fragment that can be covalently attached. Preferably, the (general) substituents R, R1-R48 are selected from the group consisting of deuterium, halogen, CN, NO₂, haloalkyl, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof with the number of carbon atoms and heteroatoms as defined above for the respective term. Examples of monovalent substituents are s2 to s71 illustrated in FIG. 2, and herein below whereby a dashed line indicates the preferred covalent link of the shown molecular fragment to the main polymacrocyclic ligand.

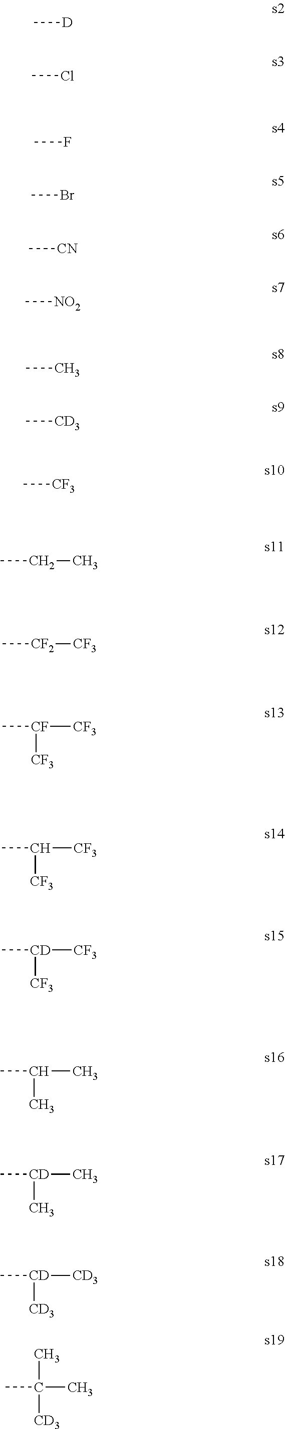

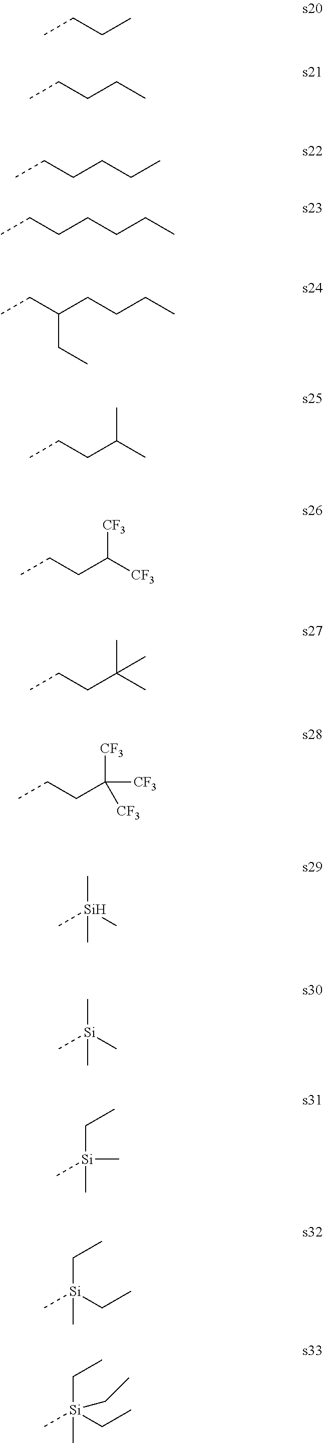

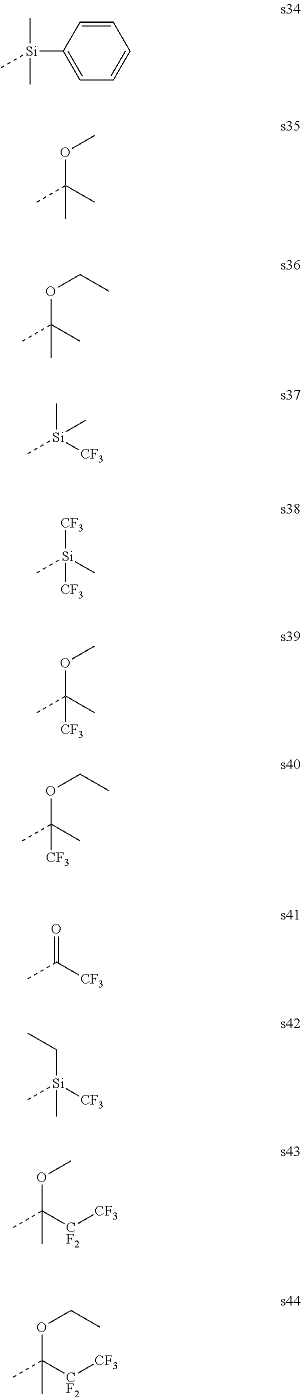

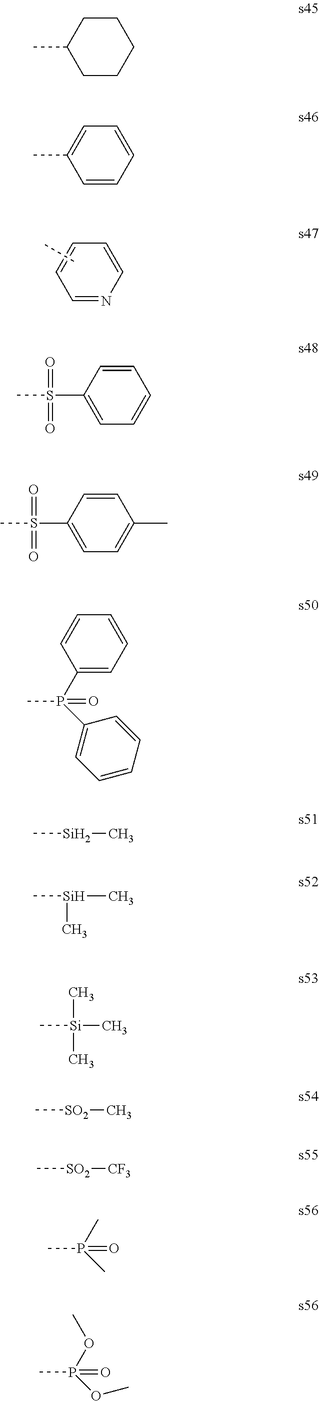

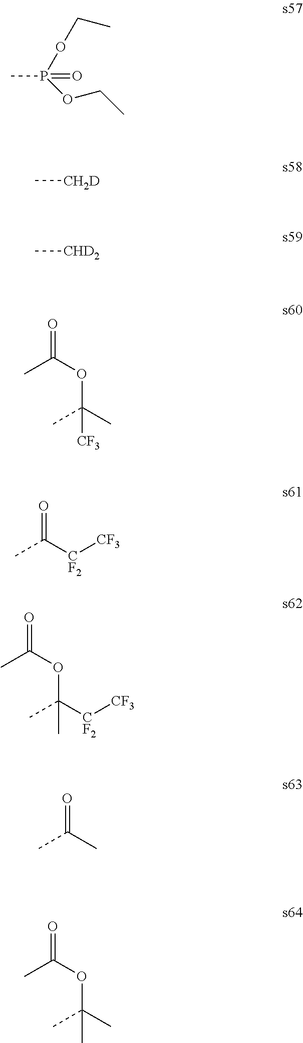

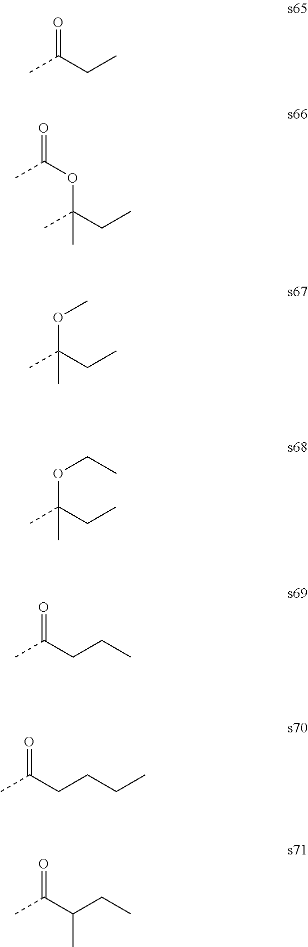

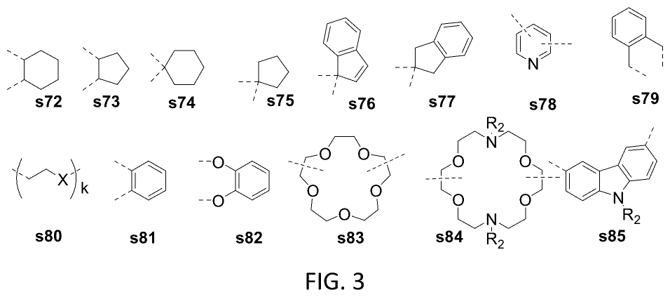

Substituents may as well be linked to themselves. The “linking to itself” may be realized by linking two R's on different C's or heteroatoms and, thus, a further macrocyclic ring is formed. For illustration purpose, formula 1g is contained in the more generic formula 1a by linking R1 with R2 and so on (assuming a=b=c=1). In this context, substitutions on the various carbon atoms in formula 1 and 2 are of course possible, but have been omitted for clarity. Alternatively, two R's at the same C may be linked (linked to itself) and, thus, realizing a spiro connection. In other words, two of the Rs bond to the same Si or C and thus give a spiro-linkage. Examples of divalent organic substituents, which however shall not limit the scope, are shown in FIG. 3 and herein below. Here, dashed lines may show the preferred covalent attachment to the macrocyclic ligand backbone.

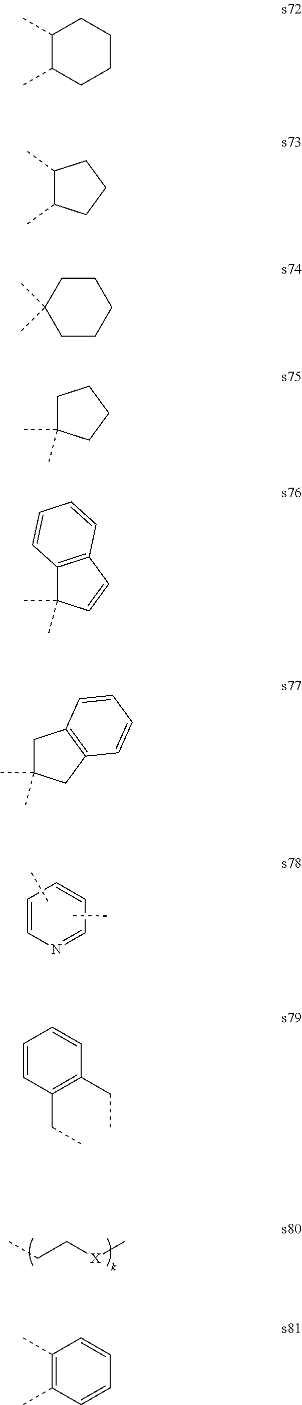

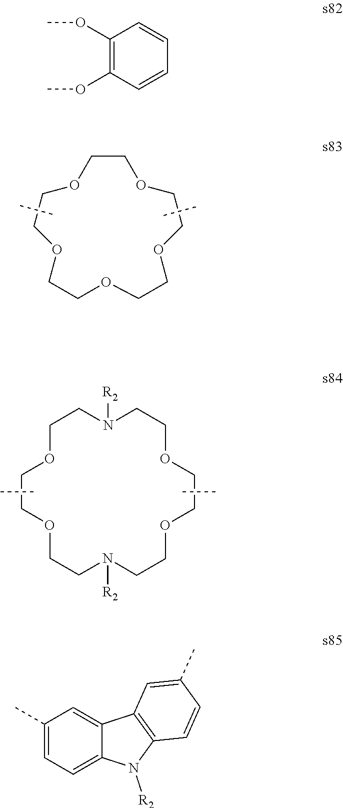

These particular types of poly macrocyclic ligands described by formula (1-1) provide a rigid and highly symmetrical, non-polarizable ligand environment for the central trivalent Cerium cation that ensures a low Stokes shift upon electrical excitation and thus prevents undesired conformational relaxation that would lead to poor Emission Quantum Yields and chemical degradation.

Whilst being symmetrical and rigid against conformational changes, the connecting units L may contain relatively chemically soft, coordinating heteroatoms, such as nitrogen. Similar, in case Y is a cyclic or macrocyclic group it again may contain soft coordinating heteroatoms. This way the natural UV emission energy of the central Cerium (III) intrametallic optical transition can readily be shifted from the UV into the deep blue spectral region as desirable for OLED applications.

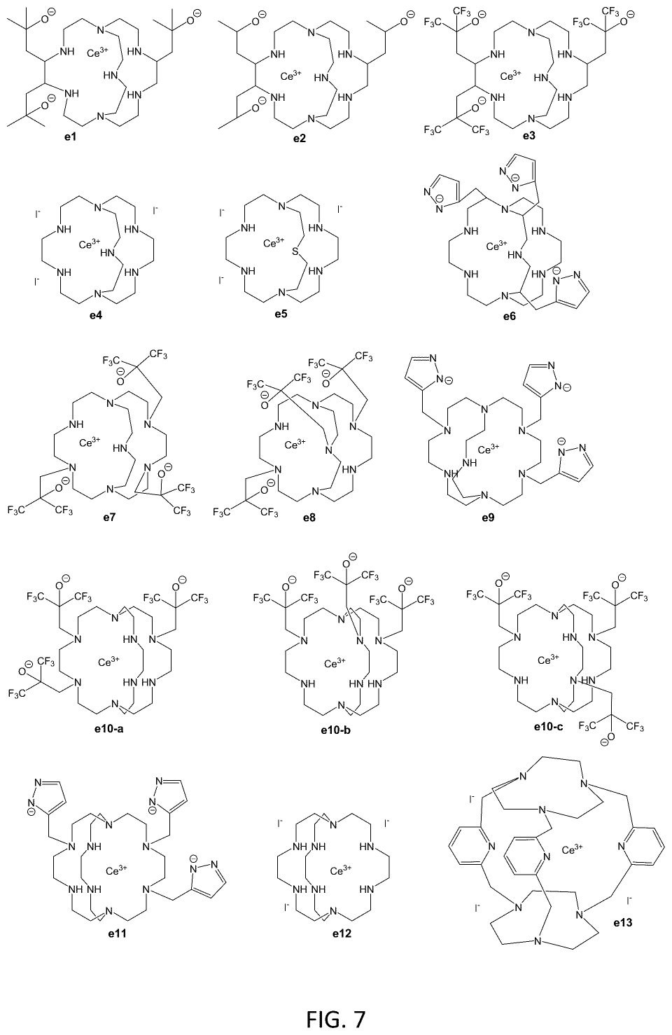

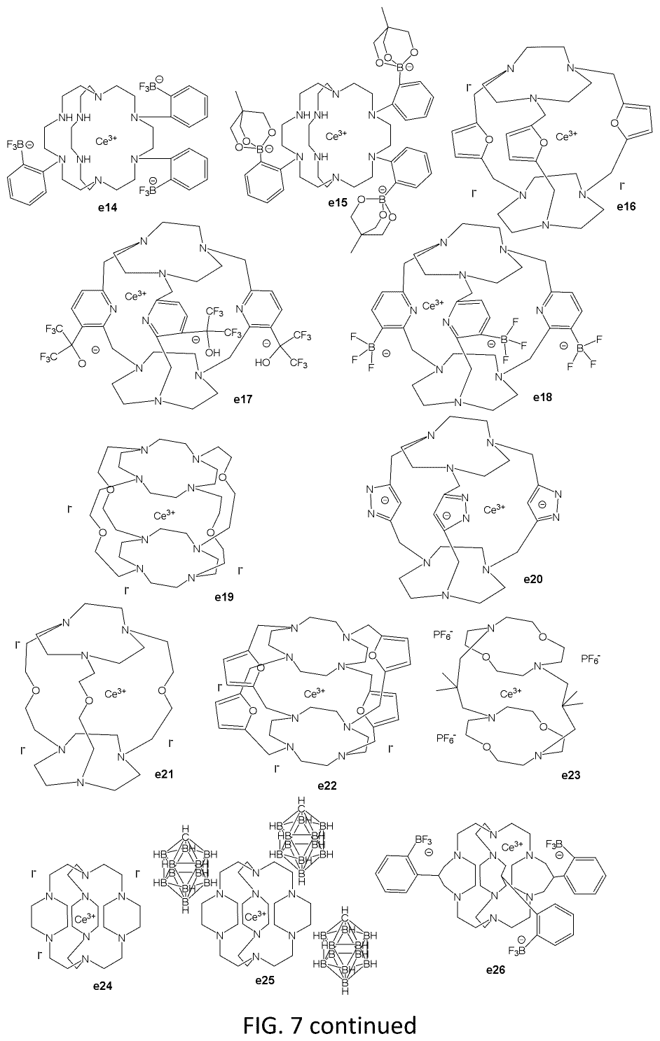

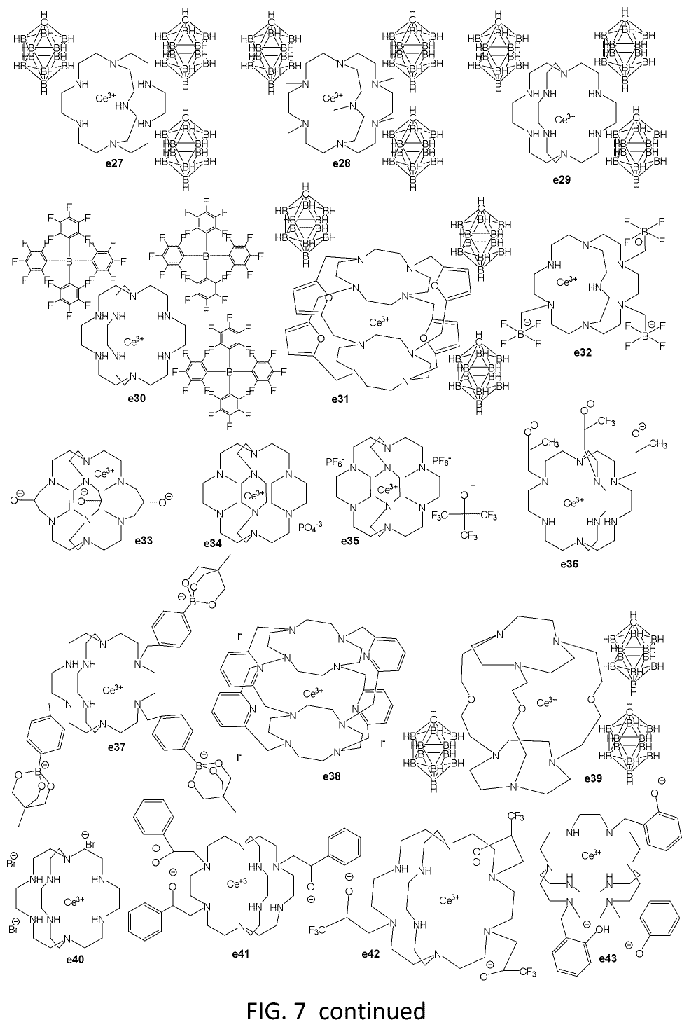

Thus, a metal organic coordination compound is provided that is highly stabilized against chemical or electrical interactions with environment, and by using soft coordinating ligands shifts the beneficial intrametallic optical transitions of the central metal cation into the desired blue spectral region. Illustratively, this is achieved by creating a nitrogen-based coordination sphere for the central Cerium cation, and simultaneously preventing interactions with the surroundings by using rigid 3D shielding ligand structure. Examples of the ligand of formula (1-1) are as follows: Preferably, n is 2, 3 or 4, more preferably 3 or 4. Preferably, Y is N or a cyclic group composed of covalently linked —N—CH₂—CH₂— and optionally —O—CH₂—CH₂— elements, that form a ring, the ring containing 2 to 4 N-atoms and 3 to 6, preferably 3 or 4 hetero atoms in total, as shown in FIG. 7. One or more hydrogen atoms in Y may be replaced by substituents, preferably C₁₋₄-alkyl groups. Preferably, not more than 4 or 3 or 2 hydrogen atoms are replaced in that way. If the ligand is as shown in FIG. 7 as e12 or e40, the anion preferably is not CF₃ SO₃⁻, more preferably an amide having a molar weight of at least 150 g/mol, more preferably at least 250 g/mol. Preferably, 1 to 3 monoanionic ligands are covalently linked with the ligand, more preferably all three ligands. In case of a ligand of type e12 or e40 in FIG. 7, preferably one of L is replaced by —CH₂CH₂—NH—CH₂CH₂— or CH₂Ch₂—O—CH₂CH₂—, with possible substitution of H in NH, as shown in FIG. 7 in e1 to e9. Preferred are ligands having a ring structure as shown in the complexes in FIG. 7, which can contain substituents as shown in the complexes in FIG. 7. Each of the shown ligands can contain or be linked with 1 to 3 monoanionic groups which are preferably as shown in FIG. 7. The ligands of formula (1-1) preferably contain 1 to 3 covalently linked monoanionic groups. In preferred ligands at least one hydrogen atom of the polycyclic groups in FIG. 7 is replaced by a substituent that is not H. In case Y is N, and each L is free of aromatic and heteroaromatic groups, then preferably at least one monoanionic ligand is covalently linked with the cyclic organic ligand, or the counterion has a molecular weight of at least 150 g/mol, more preferably at least 250 g/mol.

Thus, problems according to the related art associated with conformational relaxations in excited state as well as chemical interactions of the excited molecule with the surrounding matrix organic materials that are always present in OLED devices are elegantly circumvented. In particular, chemical degradation of the emitting atom itself during its electrical excitation is avoided, thus enhancing the device operational lifetime in OLED based consumer electronics. Cerium (III) as the electro emissive species is sufficiently heavy such that both, spin 0 and spin 1 excitations contribute to light emission by means of the heavy metal effect, ensuring high light emission efficiencies, 300% more vs currently employed fluorescent emitter materials.

In various examples, the poly macrocyclic organic ligand according to formula (1-1) that coordinates the central Cerium (III) cation may be configured to be electrically neutral. In various examples, the coordination compound may include one or two or, preferably, three singly charged anions. As example, in case the poly macrocyclic organic ligand is neutral and the metal organic coordination compound according to invention comprises three singly negatively charged non-covalently bound anions resulting in an overall charge neutral compound.

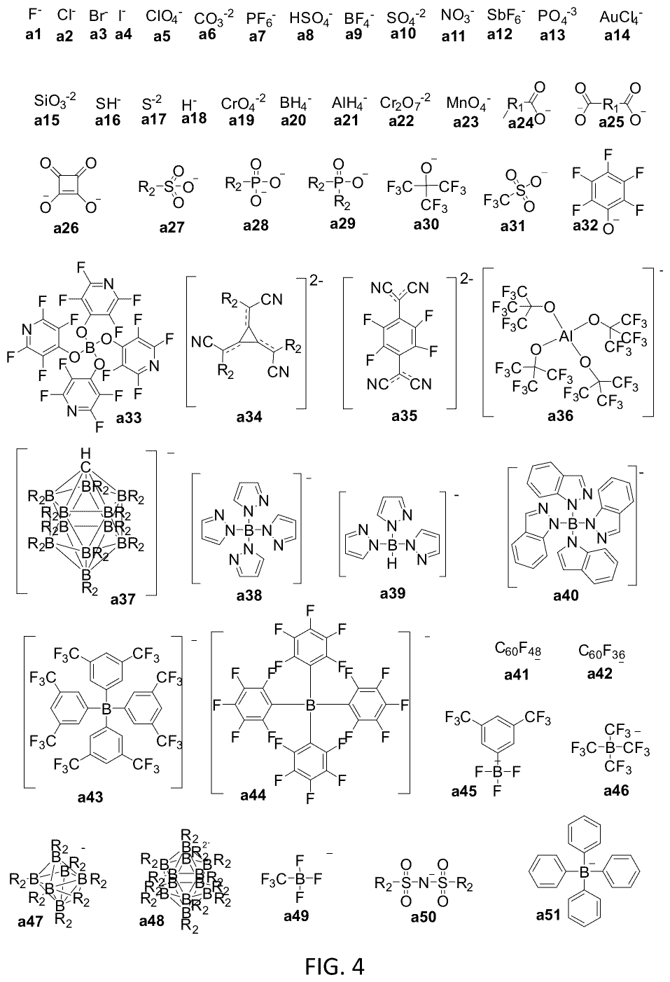

In various examples, the coordination compound may include at least one negatively charged anion, which is not covalently bound to the poly macrocyclic organic ligand. The negatively charged anion may be a small inorganic anion such as, but not limited to: F⁻, Cl⁻, Br⁻, I⁻, CIO₄⁻, BF₄⁻, PF₆⁻, SbF₆⁻, H⁻, AIH₄⁻, BH₄⁻, NO₃⁻, SO₄⁻², HSO₄⁻, SH⁻, S²⁻, CO₃²⁻, PO₄³⁻, SiO₃²⁻, AuCl₄⁻, CrO₄²⁻, Cr₂O₇²⁻, MnO₄⁻ see also a1 to a23 in FIG. 4 and herein below.

| F⁻ | Cl⁻ | Br⁻ | I⁻ | ClO₄⁻ | CO₃⁻² | PF₆⁻ | HSO₄⁻ | BF₄⁻ | SO₄⁻² | NO₃⁻ | SbF₆⁻ | PO₄⁻³ | AuCl₄⁻ |
|---|---|---|---|---|---|---|---|---|---|---|---|---|---|
| a1 | a2 | a3 | a4 | a5 | a6 | a7 | a8 | a9 | a10 | a11 | a12 | a13 | a14 |

| SiO₃⁻² | SH⁻ | S⁻² | H⁻ | CrO₄⁻² | BH₄⁻ | AlH₄⁻ | Cr₂O₇⁻² | MnO₄⁻ |
|---|---|---|---|---|---|---|---|---|
| a15 | a16 | a17 | a18 | a19 | a20 | a21 | a22 | a23 |

When an external field is applied to a layer comprising the coordination compound according to invention, for example in operative OLED device configuration, anions may drift towards oppositely charged electrodes especially when using small and non-bulky anions. Such behavior may define a so-called light emitting chemical cell.

A light emitting chemical cell may be an examples of an OLED in this description. Drift of charged species within a device including the organic electroluminescent Cerium coordination compound having a poly macrocyclic ligand may lead to very low driving voltages, which may assist to facilitate very good power efficiencies. This may be desirable for some applications, such as general illumination or signage. Yet, for other applications that require fast response times, for example flat panel displays, such ion drift may lead to time dependent OLED characteristics, which may be difficult to control, and may not be desirable as such. Therefore, the choice of the anions may depend strongly on the application.

In various examples, the coordination compound may contain comparably large and bulky organic anions. Such anions may be employed if ion drift may be not desired. Examples of such anions include fluorinated or non-fluorinated fullerene (C₆₀⁻, C₆₀F₃₆⁻, C₆₀F₄₈⁻) fluorinated aryl, carboranes, and borates, examples a24 to a51 thereof are illustrated in FIG. 4 and herein below but are not limited thereto.

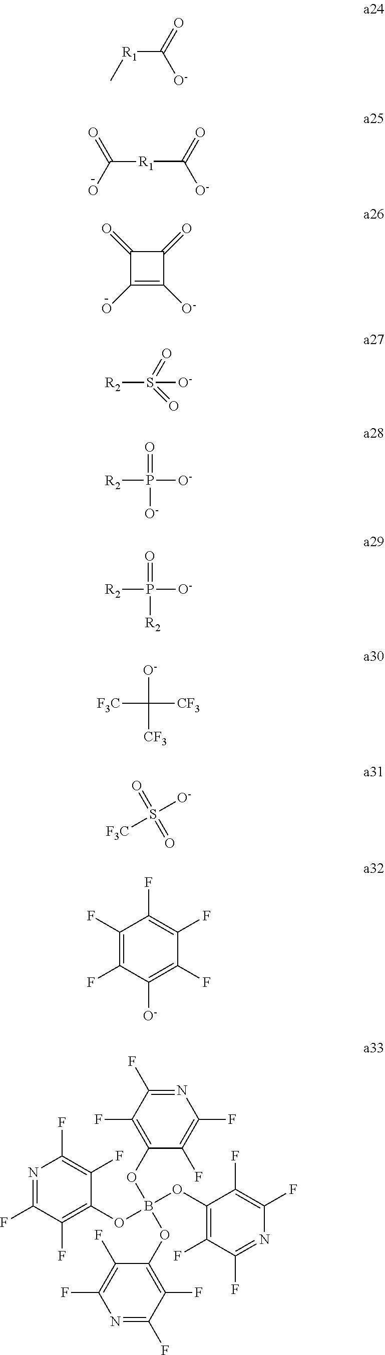

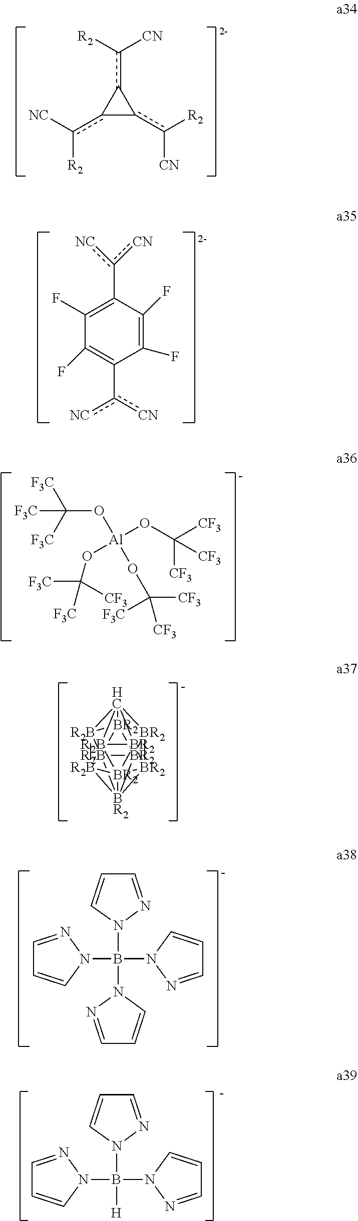

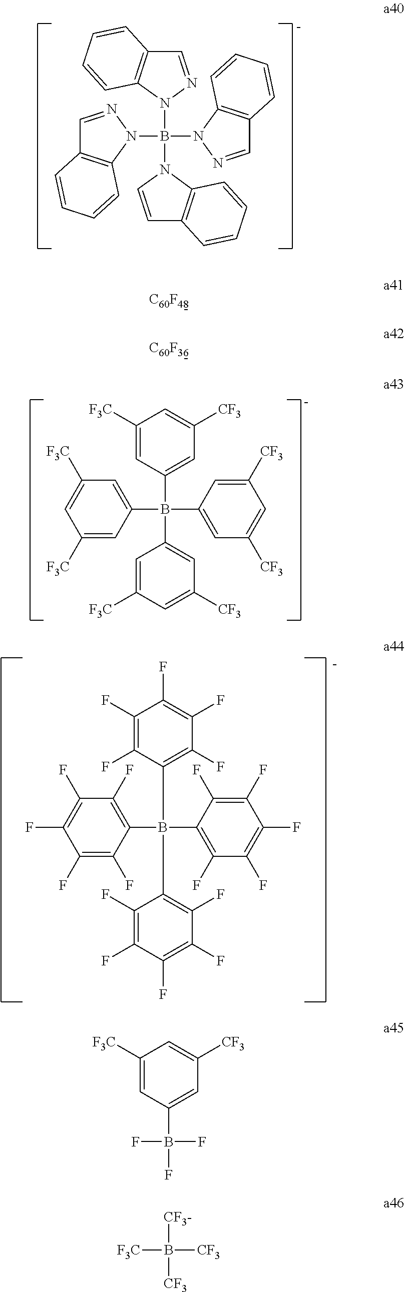

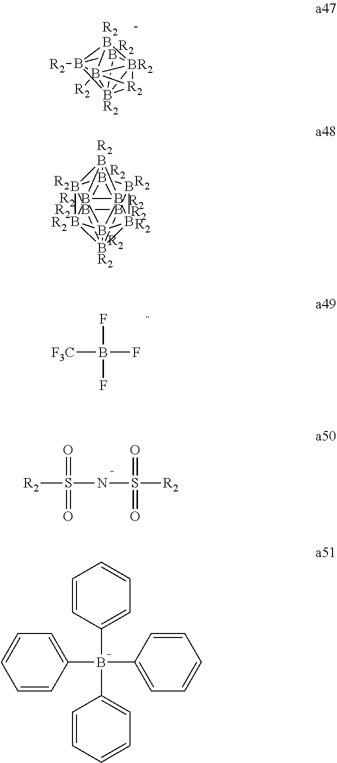

Here, R₂ preferably is hydrogen, deuterium, (preferably substituted) linear or branched C₁₋₁₀-alkyl, perfluorinated C₁₋₁₀-alkyl, partially fluorinated C₁₋₁₀-alkyl, (preferably substituted) aryl, perfluorinated aryl, partially fluorinated aryl, (preferably substituted)cycloalkyl, substituted alkenyl or substituted alkynyl.

The negatively charged anion may include more than one atom, preferably more than three atoms, and/or can have a molecular weight of at least 128 g/mol, preferably at least 150 g/mol, more preferably at least 170 g/mol, most preferably at least 250 g/mol. Preferably, CF₃ SO₃⁻ is not used as anion.

In various examples, anions without substantial absorption in the visible spectral region may be used. In case there are two or three non-covalently bound anions, they may be the same or different type.

In various examples, anions comprising Bor, known as borates, or Bor clusters, known as carboranes, may be used.

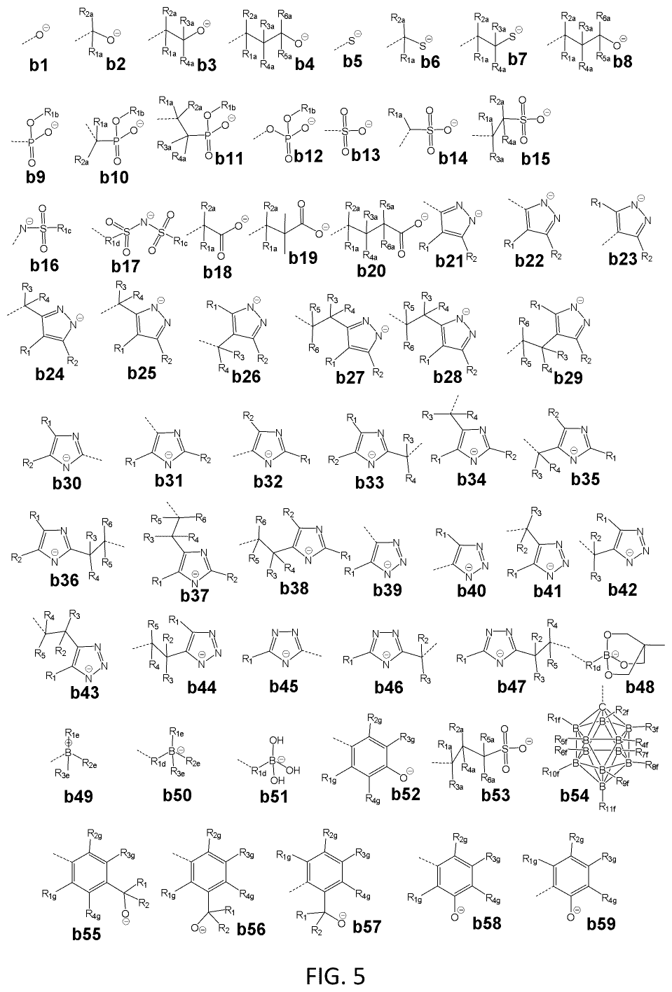

In various examples, the organic poly macrocyclic organic ligand according to formula (1-1) that coordinates to a Cerium (III) cation within the coordination compound according to invention may itself already be single or multiple negatively charged. In various examples, suitable anionic groups are covalently attached to the poly macrocyclic organic ligand. Examples of such monoanionic substitutions are illustrated in b1-b59 in FIG. 5 and herein below with.
R₁ to R₆ independently being H or organyl,
R₁ₐ to R₆ₐ independently being H, CH₃, CF₃, CN, F or CₙH₂ₙ₊₁ with n being an integer of 1 to 5,
R_{1b} being CₙH₂ₙ₊₁ with n being an integer of 1 to 5,
R_{1c} being CH₃ or CF₃,
R_{1d} being a divalent organic fragment, preferably selected from alkylene, perfluoroalkylene, which optionally can be substituted,
R₁ₑ to R₃ₑ independently being a 5-membered heteroaryl group with up to 3 heteroatoms, selected from N, S, O, which optionally can be substituted,
or a 6-membered aryl or heteroaryl group which can contain up to 4 heteroatoms, selected from N, S, O, preferably from N, and which optionally can be substituted or a group selected from CF₃, F, H, OMe, OEt.
R_{1f} to R_{11f} independently being H, Cl, Br, F, CH₃, CF₃
R_{1g} to R_{4g} independently being H, organyl, halogen, preferably F, CF₃, CN, OMe.

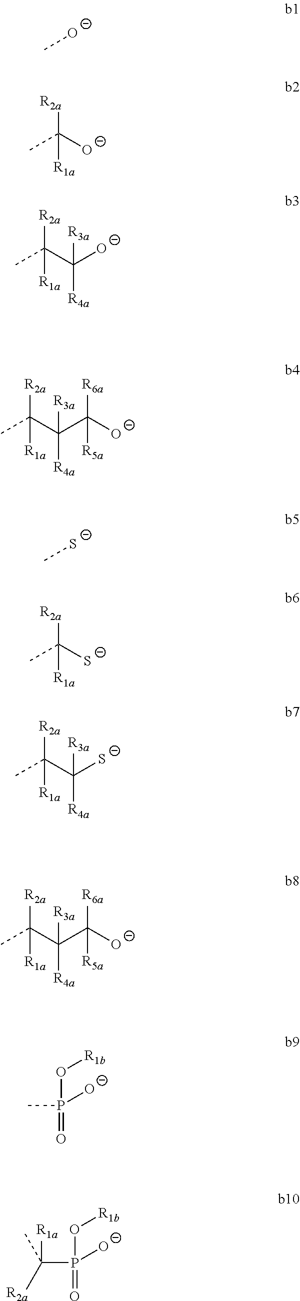

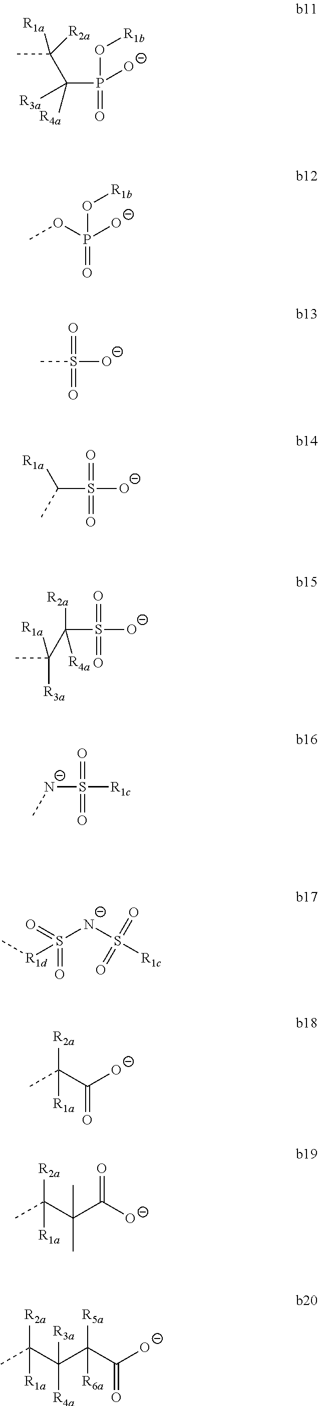

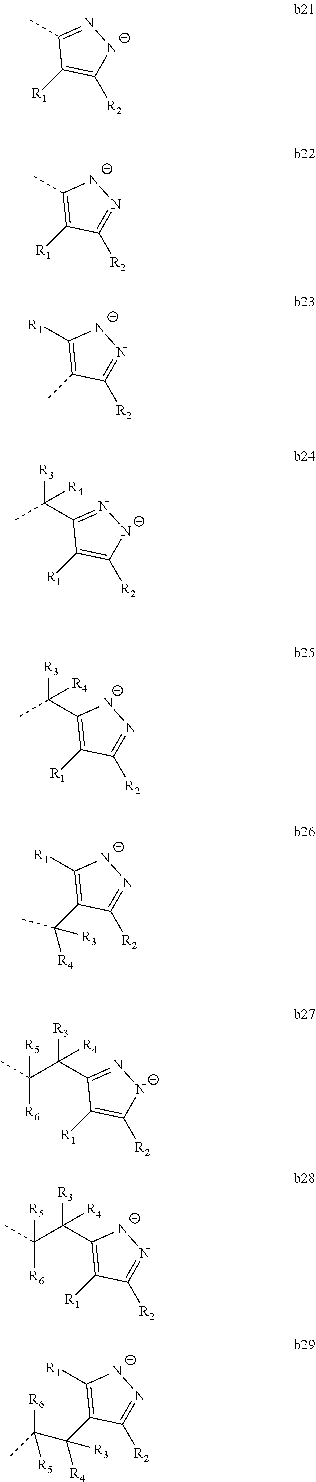

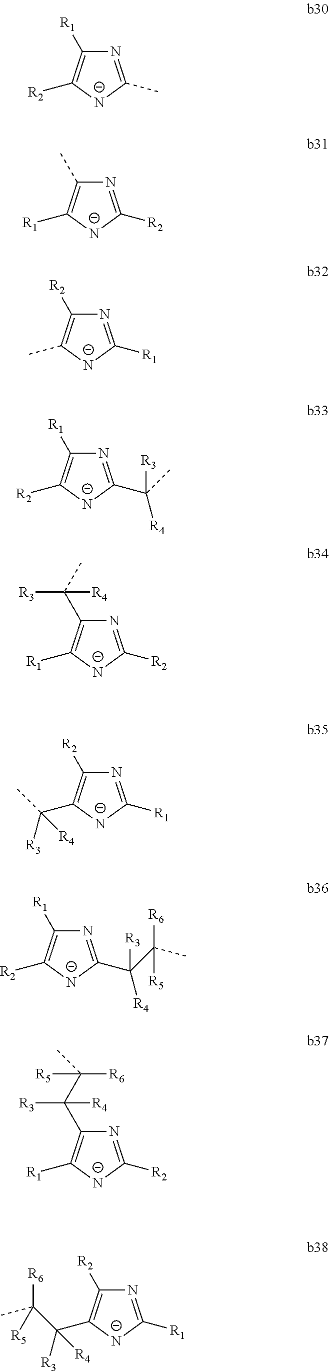

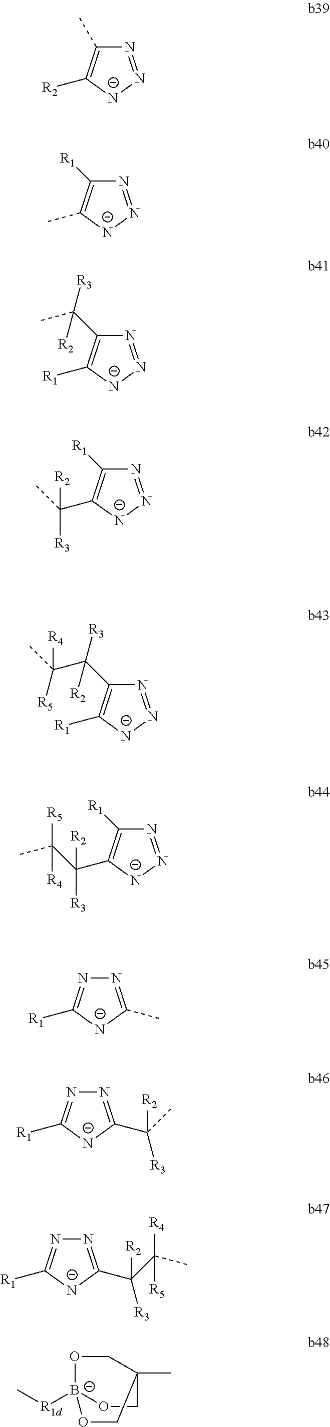

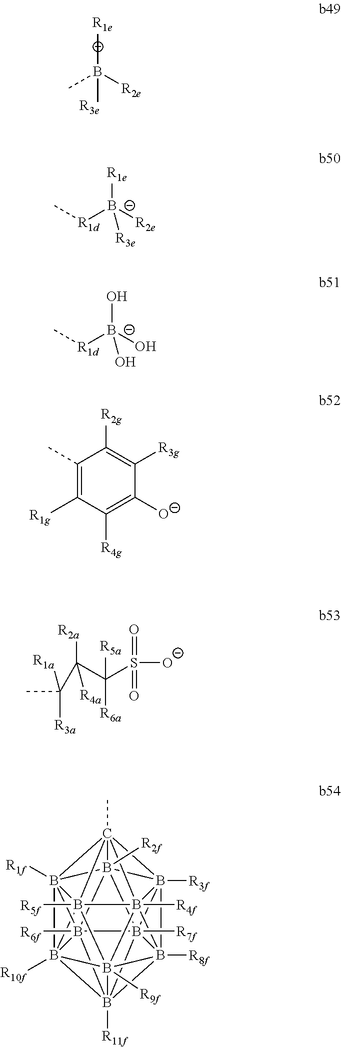

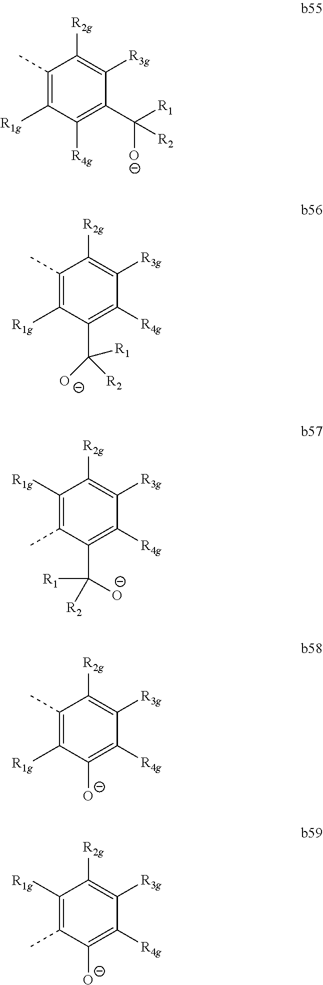

Instead of covalently attaching anionic groups, the poly macrocyclic organic ligand may as well comprise negatively charged hetero atoms, such as borates, or negatively charged aryl groups, such as pyrazolyl or cyclopentadienyl, as part of the scaffold of the poly macrocyclic organic ligand.

In preferred coordination compounds of the present invention, the poly macocyclic organic ligand according to formula (1-1) contains exactly three monoanionic groups, and therefore is threefold negatively charged and by coordinating the central Cerium (III) cation forms a complex of neutral charge. In such configuration no unbound negatively charged anions are present, therefore anion drift in device configuration under applied electric fields is elegantly circumvented.

Importantly, the mono charged anions depicted in FIG. 5 shall not limit the scope of the invention. In various examples, anionic groups covalently linked to the organic poly macrocyclic ligand may be twofold or even higher charged, for example by attaching sulfate, phosphonate, or chromate anionic groups. Similarly, the poly macrocyclic organic ligand may be negatively charged higher than 3. In such a case, a charge neutral compound may be obtained by incorporation of additional suitable organic or inorganic cations, such as ammonia, alkali, alkali earth metal cations or similar into the coordination compound according to invention.

In various examples, the coordination compound according to the invention may be used in a mixture with at least one second electrically neutral or charged organic compound. Preferable for application in electroluminescent devices, the second organic compound may have a triplet energy higher than 2.5 eV. Alternatively or in addition, the coordination compound may have a higher hole affinity compared to the second organic compound.

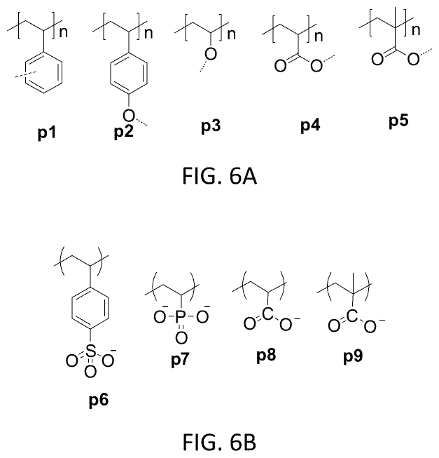

In various examples, a compound may include the coordination compound according to any of the described or illustrated examples and a polymer with a molecular weight Mn above 1000 g/mol. The coordination compound may be covalently attached to the polymer backbone. The polymer molecule may be an auxiliary organic molecule. In various examples, the poly macrocyclic organic ligand according to invention may be covalently linked to a auxiliary organic molecule or surface or polymer. Some exemplary repeat units of polymers that may bind to the poly macrocyclic ligand of the coordination compound according to invention are illustrated in FIG. 6A and herein below as p1 to p5. Here, a dashed line may represent a preferred connection to the coordination compound according to invention. It is, however, understood that those are specific examples for illustration purpose only.

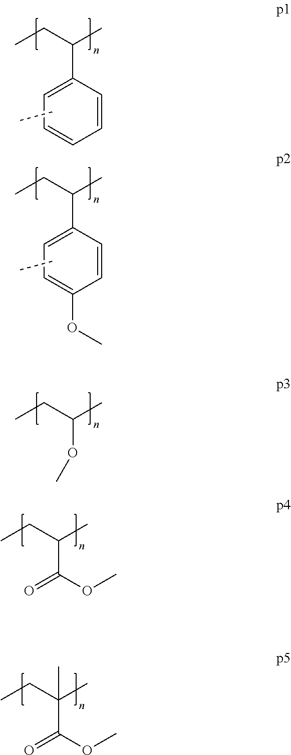

In various examples, the coordination compound including the poly macrocyclic organic ligand may contain one or more non-covalently bound anions covalently bound to a binder molecule of high molecular weight, e.g. to avoid undesired ion drift. The molecular weight of the binder molecule may be larger than 1000 g/mol. As example the binder molecule may be a polymer with more than 2 repeat units. Examples for the binder molecule being a polymer may be given by compounds p6-p9 illustrated in FIG. 6B and herein below.

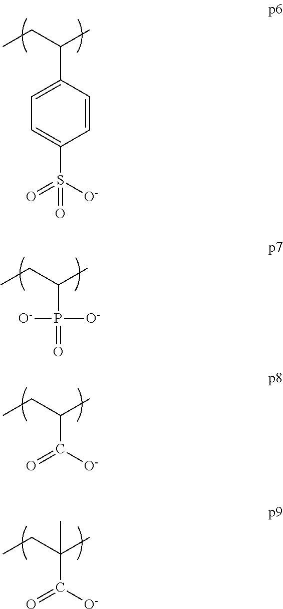

In FIG. 7, e1-e43 several metal-organic coordination compounds according to the present invention are shown for illustrative purpose. The compounds are exemplary for the concept of the present invention wherein a Cerium (III) cation is coordinated by a poly macrocyclic organic ligand according to formula (1-1).

The compounds shown have a plurality (>2) of soft donor atoms, such as nitrogen, that coordinate with the central metal cation to ensure sufficient shift of the intrametallic optical Cerium (III) transition into the blue spectral region.

It is to be understood that the poly macrocyclic organic ligand can contain further substituents at any suitable carbon or nitrogen position, specifically organyl groups, organoheteryl groups, aryl groups, heteroaryl groups, heterocyclic groups, alkenyl groups, heteroalkyl groups, hetero cycloalkyl groups, cycloalkyl groups, alkyl groups or combinations thereof. Furthermore, halogen substituents may be present in the different compounds. In FIG. 7 such possible substituents have been mostly omitted for clarity.

For illustrative purpose coordination compounds comprising only bis cyclic organic ligands are shown in examples e1-e12, 314, e15, e24-e30, e32-e37, e40-e43; while e23 is 4-cyclic; while e13, e16-18, e20, e21, e39 are 5-cyclic; and while e19, e22, e31, e38 are 6-cyclic.

Coordination compounds comprising a charge neutral poly macrocyclic organic ligand are shown in structures e4, e5, e12, e13, e16, e19, e21-e25, e27-e31, e34, e35, e38-e40. In all those cases, save e34, the charge compensation is achieved using three single charged anions, such as shown in FIG. 4, which by definition are non-covalently bound to the organic poly macrocyclic ligand, whilst in e34 a triple charged phosphate is used as charge compensating anion. Of the structures with non-covalently anions, e4, e5, e12, e21,e22, e24, e38, e40 use the halides iodide or bromide for charge compensation. In a preferred example, e23, e25, e27-e31, e34, e35, e39 contain anions with more than 3 atoms. Even more preferred, the anions used in examples e25, e27-e31, e39 have an (individual) molecular weight of greater than 128 g/mol.

Coordination compounds comprising a threefold negatively charged poly marocyclic organic ligand are shown in structures e6-11, e14, e15, e17, e18, e20, e26, e32, e33, e36, e37, e41-43. In all those exemplary structures, save e20,3 singly charged anionic groups, such as illustrated in FIG. 5, are covalently attached to the poly macrocyclic organic ligand. In case of e20, the negatively charge group is part of the scaffold of the macrocyclic ring system. In general the negatively charged anions may be covalently bound to any suitable atom of the poly macrocyclic organic ring system, for example carbon or nitrogen atoms. By example of the latter case, structural isomers are obtained. Exemplarily for e10, the three possible structural isomers by connecting via the N-position of the biscyclic organic ligand, e10a-e10c are shown. Albeit typically only one substitution pattern is arbitrarily shown, it is understood that the structures shown in e6-11, e14, e15, e17, e18, e26, e32, e33, e36, e37, e41-43 shall comprise all possible isomers.

Many of the exemplary structures shown in FIG. 7 contain aromatic building blocks as part of the poly macrocyclic organic ligands. For example, e13, e17, e18, e38 comprise pyridine; e16, e22, e31 furan; and e20 pyrazole. In each structure it is ensured that the aromatic building blocks contain no more than 8 aromatic carbon or heteroatoms, and that several such building blocks present in a single poly macrocyclic organic ligand are being separated from each other by at least one aliphatic carbon or heteroatom. This ensures an efficient conjugation break between aromatic units and as such provides poly macrocyclic organic ligands with sufficiently high triplet energy level such that the optical Cerium (III) transition has substantially intrametallic and little charge-transfer (CT) character.

In various examples, the coordination compound according to various examples may be dispersed into a matrix of suitable optical properties, such as for example a high transparency in certain desired parts of the visible spectrum. If this matrix is brought in optical contact to a light source emitting at sufficiently short wavelength, this light may be absorbed by the coordination compound and reemitted at substantially longer wavelength. A suitable light source may for example be a light emitting diode emitting light at wavelength substantially shorter than 430 nm, which may be reemitted by the coordination compound at wavelength longer than 430 nm. The matrix may have any physical dimensions. It may for example be a thin layer of 10 nm to 10,000 nm. The matrix may as well be of granular form or in form of small particles of average diameter between 10 nm and 100,000 nm. For use in optical devices, in the latter cases, the matrix may be applied inside another host material for support.

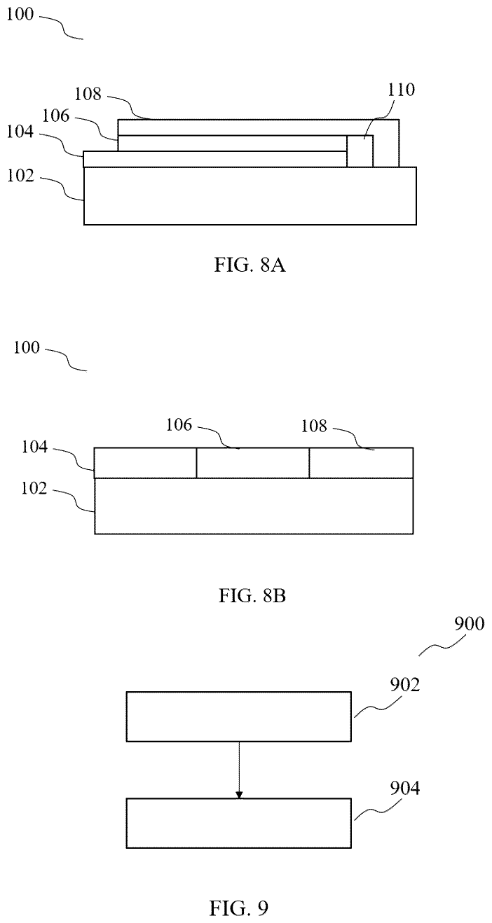

FIG. 8A and FIG. 8B illustrate examples of an organic electronic device 100 configured as optically active device. The organic electronic device may include a first electrode 104, e.g. on a substrate 102 or as the substrate; a second electrode 108; and an organic layer 106 arranged such that it is electrically interposed between the first and second electrodes 104, 108. The first and second electrodes 104, 108 may be electrically insulated from each other by an insulating structure 110, e.g. a resin or polyimide. The first and second electrodes 104, 108 may be stacked over each other (FIG. 8A) or may be arranged in a common plane (FIG. 8B).

The organic layer 106 may include the coordination compound according to any of the described or illustrated examples, e.g. as a pure compound, in a mixture or in a compound as described before.

In various examples, the organic electronic device 100 may be an optoelectronic device, the optoelectronic device being at least one of an organic light emitting diode (OLED), an organic photodetector, or a photovoltaic cell. That is, photons from an external electromagnetic field may be absorbed in the organic layer 106 and converted into current by means of an electrical field between the first and electrodes 104, 108. Such a device would be a photodiode (oPD) and its primarily use may be to sense external light. It would be an organic photovoltaic (OPV) device, if the primarily use is to convert light into current.

The organic layer 106 is arranged electrically between the first and second electrodes 104, 108 such that an electronic current may flow from the first electrode 104 through the organic layer 106 to the second electrode 108 and vice versa during operation, e.g. in light emission applications. Alternatively, in photoelectric applications, a charge carrier pair may be formed in the organic layer 106 and charge carriers of the charge carrier pair may be transported to the first and second electrodes 104, 108 respectively. In other words, in light emission applications, upon application of sufficient voltage, holes and electrons are injected from the anode and the cathode, respectively, and drift towards the organic layer 106, where charges of opposite sign recombine to form a short-lived localized excited state. The short-lived excited state may relax to the ground state thereby giving rise to light emission.

The first and second electrodes 104, 108 may be substantially unstructured layers, e.g. for general lighting applications, or may be structured, e.g. for light emitting diodes or transistors for pixels in a display application.

The organic electronic device 100 may be configured to emit substantially monochromatic light such as red, green, blue, or polychromatic light such as white. The light may be emitted through the first electrode 104 (bottom emitter), through the second electrode 108 (top emitter), or through first and second electrodes 104, 108 (bidirectional emitter). The light may as well substantially be emitted in a direction parallel to the organic layer 106 using suitable opaque electrodes 104, 108. In such a layout lasing may be achieved, and the device may be an organic laser, which, in this description, may be considered as a specific type of electroluminescent devices.

The coordination compounds according to various examples may have excellent emission properties, including a deep blue emission spectrum with short excited state lifetime. Given its high atomic weight, the optical transition of Cerium (III) may be as well widely indifferent to excitation with either spin 1 or spin 0. In other words, they are of phosphorescent type. Scientifically, phosphorescence is often exclusively used for (spin-forbidden) optical transitions from a spin 1 triplet excitation towards a spin zero singlet excitation. In this more specific definition frame, the Cerium (III) (spin-allowed) optical transition operates from a Spin ½ excited state towards a Spin ½ ground state, and is classified as a doublet. The excited state lifetime of this transition is very short-typically less than 100 ns. As such the coordination compounds according to various examples may be ideally suited for application in organic electroluminescent devices, such as organic light emitting diodes (OLED). In this description, an electroluminescent device may be any device including an organic layer disposed between and electrically connected to an anode 104/108 and a cathode 108/104. Upon application of sufficient voltage, holes and electrons may be injected from the anode 104/108 and the cathode 108/104, respectively, and drift towards the organic layer 106, where charges of opposite sign recombine to form a short-lived localized excited state. The short-lived excited state may relax to the ground state thereby giving rise to light emission. Relaxation pathways without light emission, such as thermal relaxation, may be possible too, but may be considered undesirable, as they lower the conversion efficiency of current into light of the device.

Further layers in electrical connection between the first and second electrodes 104, 108 may be formed, e.g. configured for charge carrier (electron or hole) injection, configured for charge carrier transport, configured for charge carrier blockage or configured for charge generation. Further optically functional layers, e.g. a further electroluminescent material and/or a wavelength conversion material may be formed electrically between the first and second electrodes 104, 108 and in the optical path of the organic layer 106, e.g. on top of the second electrode 108 and/or on the opposite side of the substrate 102. In addition, encapsulation structure may be formed encapsulating the electrically active area, e.g. the area in which an electrical current flows, and may be configured to reduce or avoid an intrusion of oxygen and/or water into the electrically active area. Further optically functional layers, e.g. an antireflection coating, a waveguide structure and/or an optical decoupling layer may be formed within the optical light path of the organic layer 106.

As example, hole or electron blocking layers may be used to optimize the individual hole and electron currents through the organic electronic device 100. This may be known to those skilled in the art as charge balance in order to optimize efficiency and operational stability. In various examples, dedicated hole or electron charge transport layers may be present in the organic electronic device 100 to space the emission region from the first and second electrodes 104, 108.

Examples of hole transport materials include known materials such as fluorene and derivatives thereof, aromatic amine and derivatives thereof, carbazole derivatives, and polyparaphenylene derivatives. Examples of electron transport materials include phosphine-oxide materials and derivatives thereof, oxadiazole derivatives, triazine derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and its derivative, diphenoquinone derivatives, and metal complexes of 8-hydroxyquinoline and its derivatives.

In various examples, those charge transport layers may include electrical dopant molecules or metals, or may be in contact to charge injection layers.

Any of those auxiliary layers may be fully organic or may include inorganic functional moieties. For example, charge transport layers may be made of the class of Perovskite materials. In case those auxiliary layers are composed substantially of organic materials, the used materials may be of comparably low molecular weight, below 1000 g/mol, or high molecular weight >1000 g/mol. In latter case, polymeric organic materials may be employed, for example the polymer mixture of poly(3,4-ethylenedioxythiophene) with polystyrene sulfonate known as PEDOT-PSS.

The coordination compound as illustrated or described in any of the examples may be used as a pure organic emitting layer 106 of any thickness in the range of 1 nm and 100 nm.

The organic layer 106 may in various examples include charge transport materials to improve charge transport into and through the organic layer 106. Charge transport materials may be any material that is able to transport either holes or electrons or both types of charges. In particular a charge transport material may be any aryl, or heteroaryl organic compound or any metal complex or any mixture thereof. The volume percentage of the coordination compound as a function of the combined charge transport materials may be between 0.5 and 99.5 vol % in the organic layer 106.

In various examples, the oxidation potential of the coordination compound of the organic layer 106 may be higher compared to all charge transport materials present in the organic layer 106. A wide variety of techniques on how to measure oxidation potentials have been published in the literature. However, for the purpose of various examples, the particular technique of how to measure the oxidation potential is not essential, e.g. only the relative order between the coordination compound and the charge transport materials may be of importance. Oxidation potentials may for example be deducted using quantum mechanical computing techniques based on density functional theory and experimental techniques are very well known in the art, e.g. see R.J.Cox, Photographic Sensitivity, Academic Press, 1973, Chapter 15.

The organic layer 106 may contain any other organic or inorganic material in a range of 0.1 to 99.9 vol % that are not intended to transport charges. For example, the organic layer 106 may include polymers (in a mixture or as a compound) to improve film quality and prevent crystallization. Other materials may be added to evenly space the coordination compound inside the organic layer 106.

In color conversion materials, the well-shielded and robust coordination compounds based on Cerium (III) according to various examples may allow the ease of processing using vacuum based techniques. Thus, an application of Cerium (III) metal with its favorable optical transition in organic electronic devices, such as organic photovoltaic (OPV), organic light emitting diodes (OLED), organic sensors, organic memory or organic sensors may be of advantage. In other words, the evaporation temperature of the compounds including the Cerium (3+) cation may be sufficiently low to allow for thermal vacuum processing techniques to be used. Reduced evaporation temperatures can be achieved by converting the inorganic salts including the Cerium metal in trivalent oxidation state into metal-organic coordination compounds. In such an environment the ionic character of the compound is strongly suppressed, if compared to a similar inorganic salt. Consequently, the evaporation temperature is reduced and incorporation into organic electronic devices becomes possible using state-of-the-art vacuum deposition techniques.

The excitation of the light emitting coordination compound may be electrically confined. This way, high efficiencies may be achieved. Thus, confinement layers may be formed adjacent to the organic layer 106, wherein the confinement layers may have a triplet energy Ti higher than 2.3 eV, e.g. higher than 2.5 eV, e.g. higher than 2.7 eV. Similarly, any material of the organic layer 106 (other than the electroluminescent compound) may have a triplet energy T₁ higher than 2.3 eV, e.g. higher than 2.5 eV, e.g. higher than 2.7 eV.

In various examples, the organic electronic device 100 includes two or more sub units each including at least one light emitting layer. The subunits may be stacked over each other physically separated and electrically connected by a charge generation layer or, alternatively, may be arranged side by side. The subunits may be subpixels of a pixel in a display or general lighting application. The light emitted by the subunits may be mixed to generate a light of a predetermined color. Each subunit may emit light of the same or a different color. The overall light emitted by such organic electronic device 100 may contain a narrow spectral region, such as blue, or may contain a wide spectral region such as white, or a combination thereof. The coordination compound illustrated or described in any of the examples may or may not be present in any subunit of the organic electronic device 100.

The light emitted by the organic electronic device 100 may be in optical contact to at least one optically active layer, including any optically active materials such as organic molecules or quantum dots. The optically active layer may be a spectral filter element, which may absorb part of the light emitted by the organic electronic device 100. In an example, the optically active layer may absorb at least part of the light emitted by the organic electronic device 100 and may reemit it at longer wavelength (wavelength conversion).

As example, the organic layer 106 may be configured to emit light substantially at wavelengths shorter than 430 nm and the optically active layer may be configured to substantially reemit light at wavelengths longer than 430 nm. The optically active layer may be placed in between the anode and cathode of the organic electronic device 100 or outside of it. The optically active layer may as well be part of the organic layer 106.

The organic electronic device 100 may be configured as a large area OLED device used for illumination, signage, or as a backlight. Alternatively, the organic electronic device 100 may include a plurality of OLEDs arranged in a pixilated layout (plurality of OLED pixels) that are individually connected electrically, e.g. for flat panel display applications. Here, individual pixels may have the capability of emitting light of substantially narrow spectral portions; especially of red, green, and blue. The coordination compound may or may not be present in any of the individual pixels.

The individual pixels may be configured to emit white light. Red, green, and blue spectral portions are generated by using suitable filter elements in optical contact with the pixelated OLEDs.

The OLED pixels can emit blue light and the red and green spectral portions may be generated by using a suitable color conversion element in optical contact with the OLED pixels.

The OLED pixels emit UV light and the red, green and blue spectral portions may be generated by using a suitable color conversion element in optical contact with the OLED pixels.

As example, the organic electronic device 100 may include in various examples an anode 104/108, a cathode 108/104, and the organic layer 106 disposed between the anode and the cathode. The organic layer 106 includes the metal organic coordination compound according to a described or illustrated example. The metal organic coordination compound includes a Cerium3+ cation coordinated by polycyclic organic ligand, wherein the polycyclic organic ligand comprises at least three nitrogen atoms.

The organic electronic device 100 can include an anode 104; a cathode 108; and an organic layer 106 disposed between the anode 104 and the cathode 108. The organic layer 106 includes an electroluminescent coordination compound according to various examples. The coordination compound includes a Cerium3+ cation coordinated by polycyclic organic ligand, wherein the polycyclic organic ligand itself is charge neutral and non-covalently bound anions are present for charge compensation of the Cerium3+ cation.

The organic electronic device 100 can include an anode 104; a cathode 108; and an organic layer 106 disposed between the anode 104 and the cathode 108. The organic layer 106 includes an electroluminescent coordination compound according to various examples. The coordination compound includes a Cerium3+ cation coordinated by poly macrocyclic organic ligand according to formula (1-1), wherein the poly macocyclic organic ligand itself is threefold negatively electrically charged, such that the coordination compound is electrically charge neutral and no non-covalently bound anions are part of the coordination compound.

An organic electronic device 100 according to various examples may be fabricated using a wide range of commonly used techniques, including, but not limited to, deposition of all or some layer, from gas phase vacuum deposition, solution phase, or gas phase using a carrier gas method.

Deposition via the gas phase in vacuum may be used, whereby the coordination compound may either undergo sublimation or evaporation. The transfer into the gas phase may be improved by using a carrier gas technology, whereby an inert gas that may not be deposited into the organic layer may be helping the sublimation or evaporation of the coordination compound to be deposited.

During the deposition process from gas phase, the coordination compound may as well be co-deposited with one or more material to fabricate any desired mixed layers. Such co sublimation may as well proceed from a single evaporation source using a suitable material mixture. In this case, and prior to co-sublimation a thoroughly and intimidate mixing of the coordination compound according to invention with the second material may be advantageous. To achieve this, both materials could be dissolved in a common solvent or solvent mixture followed by solvent extraction. Alternatively, both materials might be heated above melting point and dissolved into each other. For co-sublimation with the coordination compound according to invention any materials suitable to ease the sublimation of the coordination compound or to improve the layer formation or with any other benefit may be employed. In various examples, the material used for co-deposition of the coordination compound may be a charge transport material capable of transporting either electrons or holes. In various examples, the material used for co-deposition of the coordination compound may contain tri-phenyl-amine, carbazole, silane, or phosphine oxide derivatives. The material selected for co-sublimation may in its pristine, unmixed state be of evaporation or of sublimation type. In many applications, preference may be given to sublimation type materials. The co subliming material may as well fully or in in parts decompose into smaller fragments, with those fragments may or may not be part of the re condensed material comprising the coordination compound according to invention.

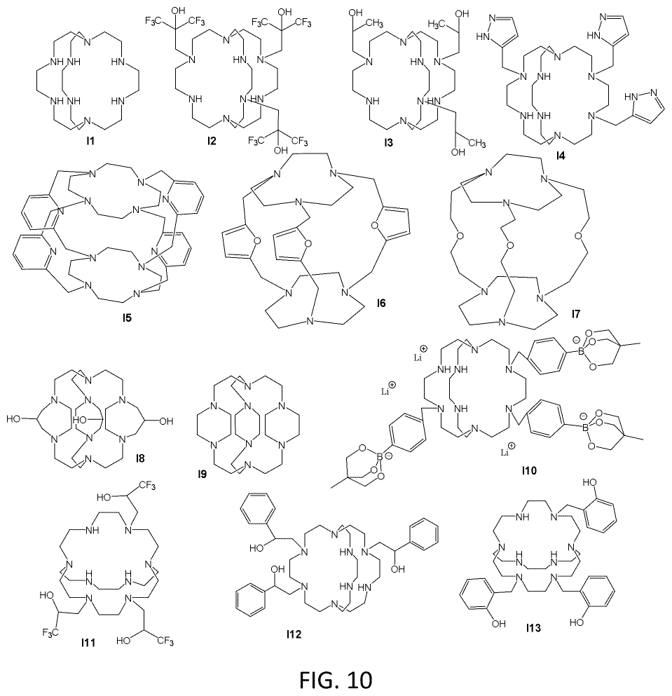

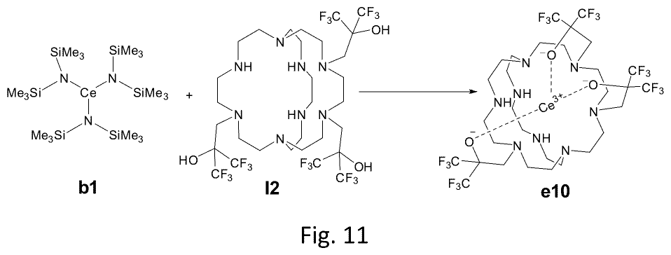

The coordination compound may be formed in-situ using gas phase deposition techniques. A flow diagram is depicted in FIG. 9 for illustration purpose. Here, the poly macrocyclic organic ligand according to formula (1-1), for example ligands illustrated in FIG. 1 or FIG. 10, excluding the Cerium (III) cation may be first (902) deposited onto a suitable substrate or other organic layer thereby forming a seed layer. Any suitable technique may be used to fabricate this seed layer. This seed layer including the poly macrocyclic organic ligand may include any other material; preferred may be organic charge transport materials, for example, suitable to achieve hole transport. Preferred may be in various examples inert organic or inorganic materials that aid the layer formation, improve its thermal stability, or improve the distribution of the polycyclic organic ligand within this seed layer. In the next deposition step (904) and sequential to forming (902) the seed layer including the poly macrocyclic organic ligand according to the formula (1-1), for example illustrated in FIG. 1 or 10, but excluding the Cerium (III) metal, a Cerium (III) containing organic or inorganic salt may be evaporated. This salt may be any charge neutral compound including Cerium (III) and one, two, or three suitable anions, or negatively charged ligands. Preferred may be three single, negatively charged inorganic anions or three single negatively charged organic ligands such as the Sil-amids depicted for illustration as compound b1 in FIG. 11. The Cerium (III) salt may as well be simultaneously co-deposited with one or more material to fabricate mixed layers. At the interface of the seed layer including the poly macrocyclic organic ligand, and because of its high thermal activation energy, the Cerium (III) salt, or parts of it, may interact with the poly macrocyclic organic ligand to form the coordination compound according to various examples in-situ. Alternatively, to use of a Cerium (III) salt, a Cerium(0) metal vapor may be used to form the coordination compound according to various examples in-situ. In the latter case, an oxidation of the Cerium into its trivalent oxidation state takes place. This reaction may be improved by the presence of suitable electron acceptor units in the seed layer, such as C60F48 or fluoro-or cyano-radialene derivatives.

Another preferred technique to fabricate layers including the coordination compound may be deposition from a liquid phase using a mixture or a single organic solvent, whereby the coordination compound according to various examples may be dissolved or forms a suspension within the organic solvent; in this description may be referred to as the ink. The ink using this deposition process, may include a wide variety of other materials apart from the coordination compound according to various examples to allow fabrication of mixed layers from solution. Additives within the ink may for example, but may not be limited to, be organic or inorganic materials capable of transporting charges, materials that improve the film formation, materials that improve the distribution of the coordination compound within a host material, organic or inorganic materials that improve the efficiency of the device, e.g. by reducing the refractive index, or materials that improve the stability of the ink, e.g. against ambient conditions. The deposition from solution may not be limited to any specific technique. Examples of the deposition from solution include spin coating, casting, dip coating, gravure coating, bar coating, roll coating, spray coating, screen printing, flexographic printing, offset printing, inkjet printing.

Various post processing techniques may be applied to improve the performance or stability of the organic electronic device. Some or all layers of the organic electronic device include functional groups capable of chemically crosslinking upon thermal or optical excitation thereby forming larger covalently bound molecules with improved physical properties. The crosslinking can take place during applied electrical field, especially such that anion drift, i.e. light emitting cell behavior, may be permanently frozen-in after the crosslinking has taken place.

The coordination compound may be formed in-situ using deposition from solution. Here, the poly macrocyclic organic ligand according to formula (1-1), for example selected from the ligands illustrated in FIG. 1 or 10, but excluding the Cerium (III) metal, may be first deposited onto a suitable substrate or other organic layer thereby forming a seed layer. Any suitable technique may be used to fabricate this seed layer. This seed layer including the polycyclic organic ligand may include any other material. Preferred may be organic charge transport materials, for example suitable to achieve hole transport for organic electronic devices. Preferred may be further additional inert organic or inorganic materials that aid the layer formation or improve its thermal stability or improve the distribution of the organic ligand within this seed layer. In a next deposition step and sequential to forming the seed layer comprising the poly macrocyclic organic ligand selected for example from, but not limited to, to structures according to formula (1-1), or ligands illustrated in FIG. 1 or 10, but excluding the Cerium (III) metal, a layer including a Cerium (III) salt may be fabricated using a solution process. The Cerium (III) salt may be any charge neutral compound that includes at least one Cerium cation in an oxidation state 3+ and one, two, or three suitable anions. The anions may as well be covalently bound to a suitable polymer or be negatively charged ligands of a suitable Cerium coordination compound. The ink including the Cerium (III) salt may as well include one or more additive to fabricate mixed layers. These additives may be organic or inorganic materials to aid charge transport, may be organic or inorganic materials that improve the efficiency of the device, or may be any material that improves the film formation. At the interface of the seed layer including the poly macrocyclic organic ligand, the solubilized Cerium (III) cation will interact with the poly macrocyclic organic ligand to form the coordination compound according to various examples in-situ.

The (electroluminescent) coordination compound according to various examples may thus be extremely stable and as such ideally suited for a large variety of processing methods and applications.

For one or more aspects, at least one of the components set forth in one or more of the preceding FIGS. may be configured to perform one or more operations, techniques, processes, and/or methods as set forth in the example section below.

Summarizing, the invention concerns a metal-organic coordination compound, wherein the coordination compound comprises at least one Cerium (III) coordinated by a poly macrocyclic organic ligand having the formula 1-1:

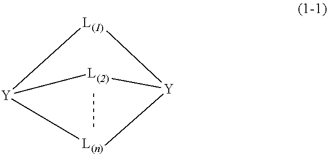

wherein n is an integer of at least 3,
Y at each occurrence is independently a n-valent group containing 1 to 30 atoms,
L at each of its n occurrence is independently a divalent organic group, wherein L has a sequence of at least 3 atoms, preferably at least 5 atoms directly connecting both Y in case that Y is a cyclic group connected with each L via different ring atoms of Y,
wherein L has a sequence of at least 5 atoms directly connecting both Y in case that Y is connected with each L via the same atom of Y,
   wherein the poly macrocyclic organic ligand comprises at least three nitrogen atoms. The sequence of at least 3 or 5, respectively, connecting atoms is the shortest sequence of connecting atoms. There may be longer sequences of connecting atoms in case that L contains a carbocyclic or heterocyclic ring forming part of the chain of connecting atoms.

Examples of the invention are summarized herein below as follows.

According to an example:
each Y is independently selected from B, B-R, N, P, C-R, or.
each Y is independently selected from 3- to 15-membered, preferably 6- to 12-membered cyclic organic groups that can contain one to four hetero atoms selected from N, B, P, O, S, and is connected with each L via non-neighboring carbon or hetero atoms, in particular via non-neighboring N atoms,
   wherein R is hydrogen or any covalently bound substituent being identical or different in each occurrence.

According to an example, the poly macrocyclic organic ligand is either non-aromatic (aliphatic) or comprises aromatic building blocks containing at most 8 aromatic carbon or hetero atoms, whereby various aromatic building blocks are being separated from each other by at least one aliphatic carbon or hetero atom.

According to an example, each Y can be a macrocyclic organic group having a sequence of ring atoms composed of sub-sequences —N—C—C— or —O—C—C—. The sequence of ring atoms in Y can be composed of 3 or 4 of the sub-sequences. The sequence of ring atoms is the shortest sequence of ring atoms in case that Y contains one or more carbocyclic or heterocyclic rings forming part of the macrocycle.

According to an example, Y can be a macrocyclic organic group comprising 9 to 12 ring atoms containing 3 or 4 hetero atoms selected from N or O, preferably N. In an example, Y can be connected with each L via a non-neighboring N atom.

According to an example, each Y can be N.

According to an example, each L can have a shortest sequence of atoms composed of sub-sequences —N—C—C— or —O—C—C—.

According to an example, the poly macrocyclic organic ligand can be threefold negatively charged and in combination with a trivalent Cerium cation forms a complex of neutral charge. The poly macrocyclic organic ligand may be threefold negatively charged by containing groups b1-b59.

According to an example, the coordination compound can comprise at least one anion that is not covalently linked to the poly macrocyclic organic ligand. Examples of negatively charged anions are a1-a51.

According to an example, the at least one anion that is not covalently linked to the poly macrocyclic organic ligand comprises >3 atoms and/or can have a mass of >128 g/mol. Examples of such anions are a24-a51.

According to an example, the coordination compound comprises exactly three singly negatively charged anions.

According to an example, the singly negatively charged anion can comprise bor. Examples of such anions are a9, a20, a33, a37-a40, a43-a49 and a51.

According to an example, the poly macrocyclic organic ligand is a bis cyclic cryptand.

According to various examples, the poly macrocyclic organic ligand can have a structure according to formula 1a-1g:

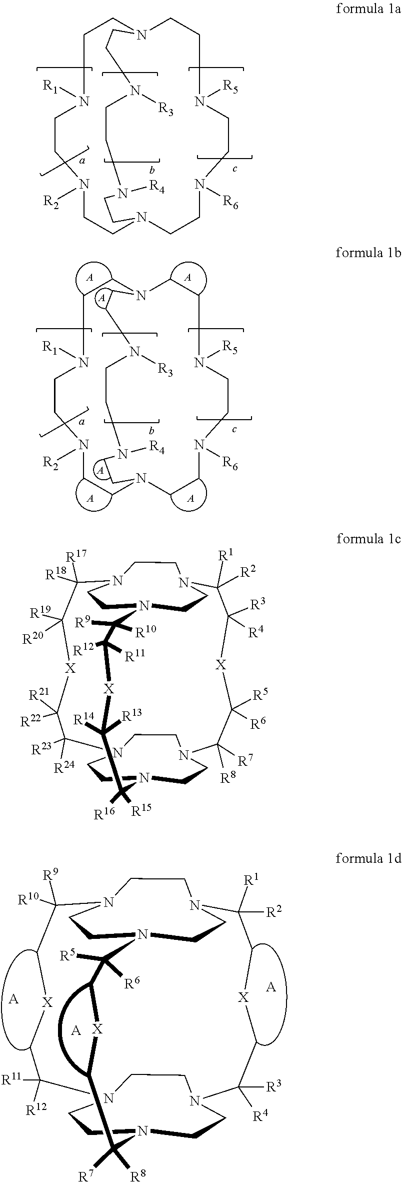

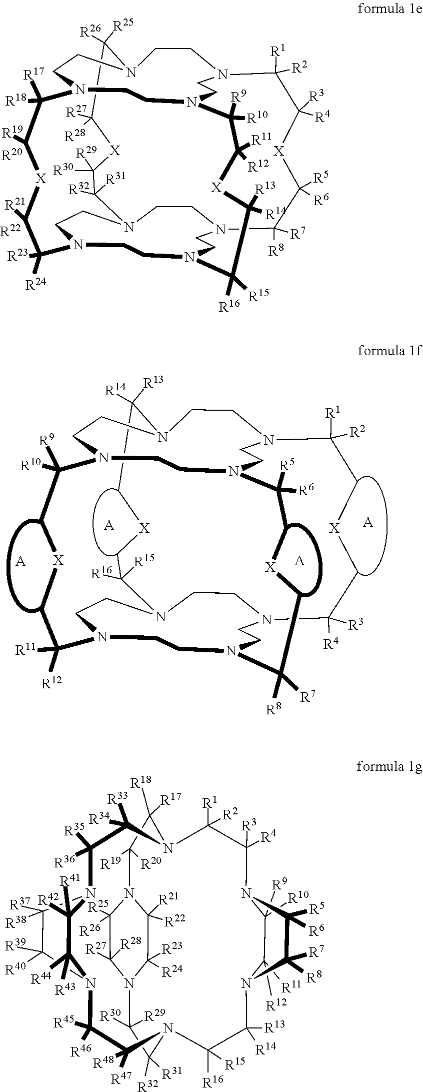

wherein
R₁-R₄₈ independently in each occurrence represent hydrogen, deuterium, Cl, F, Br, CN, NO₂ or an organyl group,
a, b, c in formula 1a and 1b are each independently an integer of 0 or more,

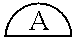

divalent cyclic organic group, wherein one or more of the ring-forming carbon atoms can be substituted or can be part of a cyclic group,
X in formula 1c, and 1e is independently in each occurrence selected from B-R, N-R, P-R, C-R-R, O, S
X in formula 1d, and 1f is independently in each occurrence selected from B, B-R, N, N-R, P, P-R, C-R, C-R-R, O, S
wherein R is hydrogen or deuterium or any covalently bound substituent being identical or different in each occurrence.

Formula 1a, 1b and 1g represent bis macrocyclic structures, formula 1c, 1d represent penta macrocyclic structures and formula 1e, 1f represent hexa macrocyclic structures.

Examples of R₁-R₄₈ are monovalent substituents s2 to s71 or divalent organic substituents s72 to s85 representing two of R₁-R₄₈ at the same atom or two different atoms. Threefold negatively charged poly macrocyclic organic ligands of formula 1a-1g may contain groups b1-b59 as one or more (up to 3) of R₁-R₄₈.

According to an example, the poly macrocyclic organic ligand comprises monoanionic groups, with the monoanionic groups having the structure —CH₂—CRR—X with X being a monoanionic atom selected from O— and S—,

or the monoanionic group has the structure

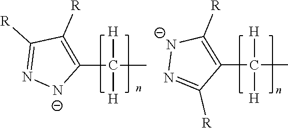

with n being an integer equal to one or more and with each R independently being H, substituted or unsubstituted C₁-12-alkyl, substituted or unsubstituted aryl, halogen, and wherein two groups R can be covalently linked to form a cyclic group.

According to an example, the coordination compound can have the structure

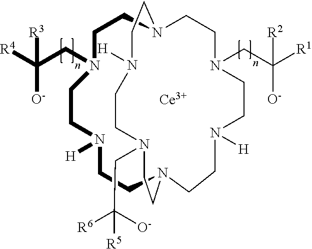

with n being an integer equal to one or more and with R₁-R₆ independently being H, deuterium, substituted or unsubstituted C₁-12-alkyl, substituted or unsubstituted aryl, halogen, and wherein two groups R can be covalently linked to form a cyclic group.

The invention further concerns a mixture comprising
a second electrically neutral organic compound, and
the coordination compound,
wherein the coordination compound is imbedded into the at least one second electrically neutral organic compound,
wherein the second organic compound has a triplet energy higher than 2.5 eV, preferably higher than 2.6 eV and even more preferably higher than 2.7 eV and/or wherein the coordination compound has a higher hole affinity compared to the second organic compound.

The invention further relates to a polymeric compound, the coordination compound, and a polymer with a molecular weight Mn above 1000 g/mol, wherein the coordination compound is covalently attached to the polymer backbone.

The invention further relates to an organic electronic device, comprising:
a first electrode;
a second electrode; and
an organic layer arranged such that it is electrically interposed between the first and second electrodes, wherein the organic layer comprises the coordination compound according to any examples, the mixture according to the example or the polymeric compound.

The organic electronic device is an optoelectronic device, the optoelectronic device being at least one of an organic light emitting diode, an organic photodetector, or a photovoltaic cell.

The invention further relates to a method of forming an organic device, the method comprising:
forming a layer comprising the coordination compound according to any example above, of the mixture above or the polymeric compound described,
wherein the layer is deposited from a gas phase, in particular using an evaporation and/or sublimation and/or carrier gas process, and/or by a solution-based process.

For forming the layer a step can comprise forming a first layer comprising the organic poly macrocyclic ligand and forming a second layer directly in contact with the first layer, wherein the second layer comprises a Cerium salt.

For forming the layer the step can comprise evaporation and/or sublimation of the organic poly macrocyclic ligand and evaporation and/or sublimation of a Cerium salt whereby the evaporation or sublimation proceeds sequentially and/or simultaneously (in latter case co-evaporation).

The coordination compound can be transferred into gas phase from a blend of the coordination compound with at least one second material, with the second material is present in the blend between 1 and 99 vol %.

The invention further concerns, the use of a metal-organic coordination or the polymeric compound in an organic electronic device, preferably an organic light emitting device.

Any of the above-described examples may be combined with any other example (or combination of examples), unless explicitly stated otherwise. The foregoing description of one or more implementations provides illustration and description, but is not intended to be exhaustive or to limit the scope of aspects to the precise form disclosed. Modifications and variations are possible in light of the above teachings or may be acquired from practice of various aspects. Any of the above-described examples may be combined with any other example (or combination of examples), unless explicitly stated otherwise. The foregoing description of one or more implementations provides illustration and description, but is not intended to be exhaustive or to limit the scope of aspects to the precise form disclosed. Modifications and variations are possible in light of the above teachings or may be acquired from practice of various aspects.

While the invention has been particularly shown and described with reference to specific aspects, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

Some specific examples according to the invention are described hereafter, which shall however not limit the overall scope of the invention. First the chemical synthesis of several Cerium (III) coordination compounds according to inventions is described. In a second set of examples results of sublimation experiments using a co-sublimation are described in detailed. Finally, electroluminescent devices using coordination compounds according to invention are described.

As a general remark with respect to chemical synthesis, all reactions were carried out under protective gas atmosphere using anhydrous solvents unless stated otherwise. Starting compound were either synthesized using known procedure or purchased from a commercial source (Table 1).

TABLE 1
Starting compounds

| Structure | Name | Abbreviation | Source |
|---|---|---|---|
| 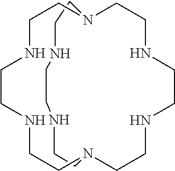 [SMILES: C1CNCCN2CCNCCNCCN(CCN1)CCNCCNCC2] | 1,4,7,10,13,16,21,24- octaazabicyclo[8.8.8]hexacosane | L1 | Prepared according to known procedure (Redko et al. SYNTHESIS 2006, 5, 0759-0761) |
| 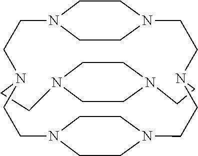 [SMILES: C1CN2CCN1CCN1CCN3CCN(CC3)CCN(CC2)CCN2CCN(CC2)CC1] | 1,4,7,10,13,16,21,24- octaazapentacyclo[8.8.8.24,7.213,- 16.221,24]dotriacontane | I9 | Prepared according to known procedure (Redko et al. JACS, 2005, 127, 12416- 12422) |
| 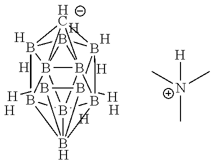 | Trimethylammonium carbadodecaborate | TMA- CB₁₁H₁₂ | Obtained from Katchem spol. s r.o (CAS: 108608-25- 9, product Nr. 212) |
| 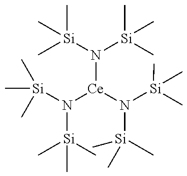 | Tris(bis(trimethylsilyl)amino)cerium | b1 | Obtained from Alfa Aesar (CAS: 41836-21-9, product Nr. 44362.06) |

Hereafter the synthesis of the polycyclic organic ligands is described. For further illustration, all ligands described in this examples are depicted in as I1-I13 in FIG. 10.

### Synthesis of Hydroxyethyl-Derivatives of I1

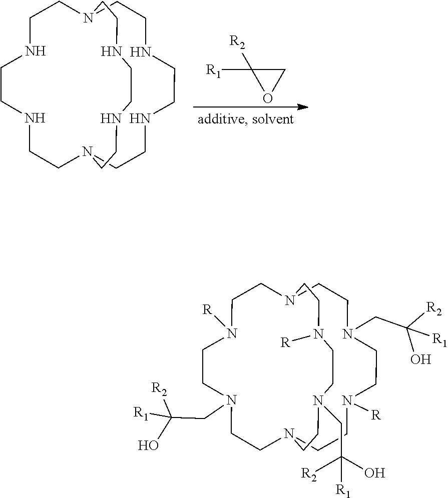

Method a: Octaamine I1 (1.4 mmol, 1.0 eq.) and dry THF (25 mL) were placed in a double neck round bottom flask which was purged with Ar. A solution of fluoromethyl-oxirane in THF (100 ml, 4.2 mmol, 3.0 eq.) was added dropwise at 0° C. and the mixture was stirred overnight at 0° C. before being allowed to reach room temperature over a course of 2 hours. All volatiles were removed under reduced pressure. The product was crystallised from a mixture of toluene/hexane, yielding the product as a white powder.

Method b: Octaamine (5.4 mmol, 1.0 eq.) was placed into a reactor, melted and degassed in vacuum at 150° C. The reactor was filled with N2 gas. The amine was cooled down to RT and 15 ml of oxygen-free anhydrous acetonitrile was added to dissolve the solid. A solution of oxirane (10.8 mmol, 3.0 eq.) in 5 ml of oxygen-free anhydrous acetonitrile was dropwise added, followed by addition of lithium tetrafluoroborate (10.79 mmol, 3.0 eq.) in 10 ml anhydrous oxygen-free acetonitrile. The mixture was stirred under reflux until the reaction was completed as indicated by ESI-MS. The solvent was evaporated to dryness and the solid residue extracted with toluene. Evaporation of the solvent under reduced pressure yielded a crude mixture of multiple substituted amines and their constitutional isomers. The mixture was separated by column chromatography to give the 3-fold substituted product (Al2O3 neutral, ethylacetate-methanol).

TABLE 2
Ligands prepared using above described methods a and b:

| | | | Yield, | NMR | ESI- |
|---|---|---|---|---|---|
| Code | Structure/Name | Method | % | (solvents) | MS |
| I2 | 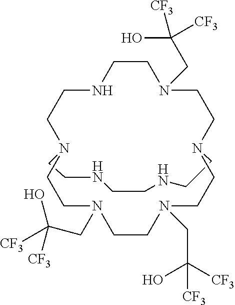 [SMILES: OC(CN1CCNCCN2CCNCCNCCN(CC1)CCN(CC(O)(C(F)(F)F)C(F)(F)F)CCN(CC(O)(C(F)(F)F)C(F)(F)F)CC2)(C(F)(F)F)C(F)(F)F] | a | 86 | 2.2 − 2.8(m,b) (60 MHz, CDCl3) | 911.7 (M + H)⁺ |
| | 2,2',2″-((1,4,7,10,13,16,21,24- | | | | |
| | octaazabicyclo[8.8.8]hexacosane- | | | | |
| | 4,7,13- | | | | |
| | triyl)tris(methylene))tris(1,1,1,3,3,3- | | | | |
| | hexafluoropropan-2-ol) | | | | |
| I11 | 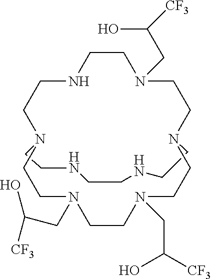 [SMILES: OC(CN1CCNCCN2CCNCCNCCN(CC1)CCN(CC(O)C(F)(F)F)CCN(CC(O)C(F)(F)F)CC2)C(F)(F)F] | a | 74 | 2.2 − 2.8(m,b) 4.6 (b) (60 MHz, CDCl3) | 707.5 (M + H)⁺ |
| | (3,3′,3″-(1,4,7,10,13,16,21,24- | | | | |
| | octaazabicyclo[8.8.8]hexacosane- | | | | |
| | 4,13,21-triyl)tris(1,1,1- | | | | |
| | trifluoropropan-2-ol)) | | | | |
| I3 | 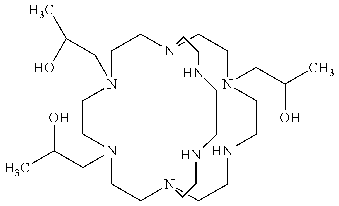 [SMILES: CC(O)CN1CCNCCN2CCNCCNCCN(CC1)CCN(CC(C)O)CCN(CC(C)O)CC2] | b | 66 | 4,12-3,74 (b), 3,71-2,52 (b), 2,50-2,20 (b), 1,97-1,01 (b) (CDCl3, 60 MHz) | 545.5 (M + H)⁺ |
| | 1,1′,1″-(1,4,7,10,13,16,21,24- | | | | |
| | octaazabicyclo[8.8.8]hexacosane- | | | | |
| | 4,7,13-triyl)tris(propan-2-ol) | | | | |
| I12 | 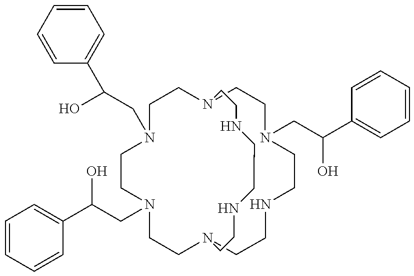 [SMILES: OC(CN1CCNCCN2CCNCCNCCN(CC1)CCN(CC(O)c1ccccc1)CCN(CC(O)c1ccccc1)CC2)c1ccccc1] | b | 66 | 7,44-7,12 (b), 4,82-4,56 (b), 4,12-3,74 (b), 3,71-2,52 (b), 2,50-2,20 (b) (CDCl3, 60 MHz) | 731.5 (M + H)⁺ |
| | 2,2',2″-(1,4,7,10,13,16,21,24- | | | | |
| | octaazabicyclo[8.8.8]hexacosane- | | | | |
| | 4,7,13-triyl)tris(1-phenylethan-1-ol) | | | | |

Synthesis of 4,7,21-tris((1H-pyrazol-3-yl)methyl)-1,4,7,10,13,16,21,24-octaazabicyclo [8.8.8] hexacosane (14)

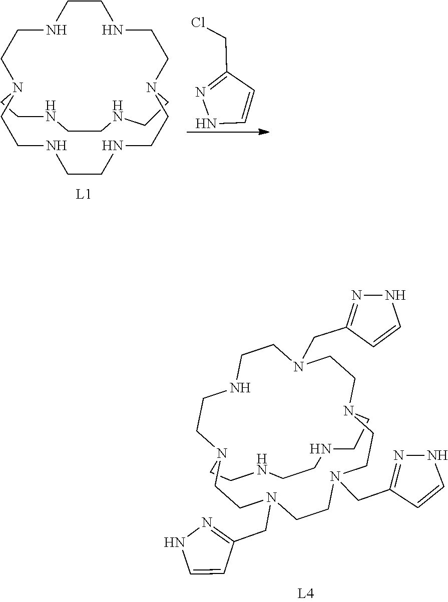

A double neck round bottom flask was charged with 1.4 mmol (1.0 eq.) of octaamine I1 and THF (25 mL). 3-(Chloromethyl)-1H-pyrazole (4.2 mmol, 3.0 eq.) was added in one portion and the mixture was refluxed for 5 hours. The precipitate was filtered, washed with THF, dissolved in water and passed through Amberlite IRA-410 (OH) anion exchange resin. The solution was concentrated and the residue extracted with toluene. Toluene was removed under reduced pressure to yield the product.

Yield: 72%; 1H NMR (300 MHZ, CDCl3): δ[ppm]=7.42 (d, 3H, Ar), 6.04 (d, 3H, Ar), 3.56 (m, 6H, CH2Ar), 2.33-2.76 (m, 36H); MS (ESI): m/z=611.4 (M+H)+

### Synthesis of 1,4,8,11,14,18,23,27-Octaazapentacyclo [9.9.9.24,8.214,18.223,27] pentatriacontane-6,16,25-triol (18)

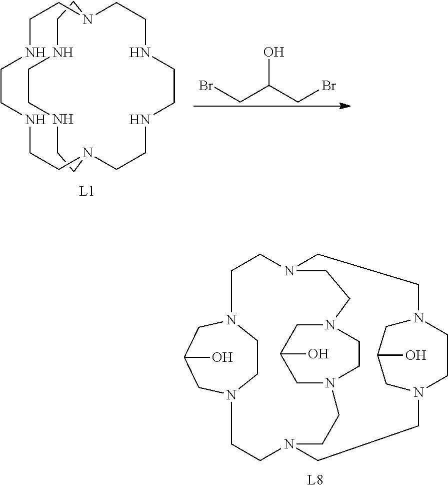

A double neck round bottom flask was charged with 1.4 mmol (1.0 eq.) of octaamine I1 and THF (250 mL). 1,3-Dibromopropan-2-ol (4.2 mmol, 3.0 eq.) was added in one portion and the mixture was refluxed for 5 days. The solvent was removed under reduced pressure. The precipitate was dissolved in MeOH and passed through Amberlite IRA-410 (OH) anion exchange resin. MeOH was removed under reduced pressure to yield the product.

Yield: 23%; 1H NMR (300 MHZ, CDCl3): δ[ppm]=2.00-3.82 (m, 51H); MS (ESI): m/z=539.2 (M+H)+

### Synthesis of Trimethylammonium 1,1′,1″-(((1,4,7,10,13,16,21,24-octaazabicyclo [8.8.8] hexacosane-4,7,13-triyl)tris(methylene))tris(benzene-4,1-diyl)} tris(4-methyl-2,6,7-trioxa-1-borabicyclo [2.2.2] octan-1-uide) (110)

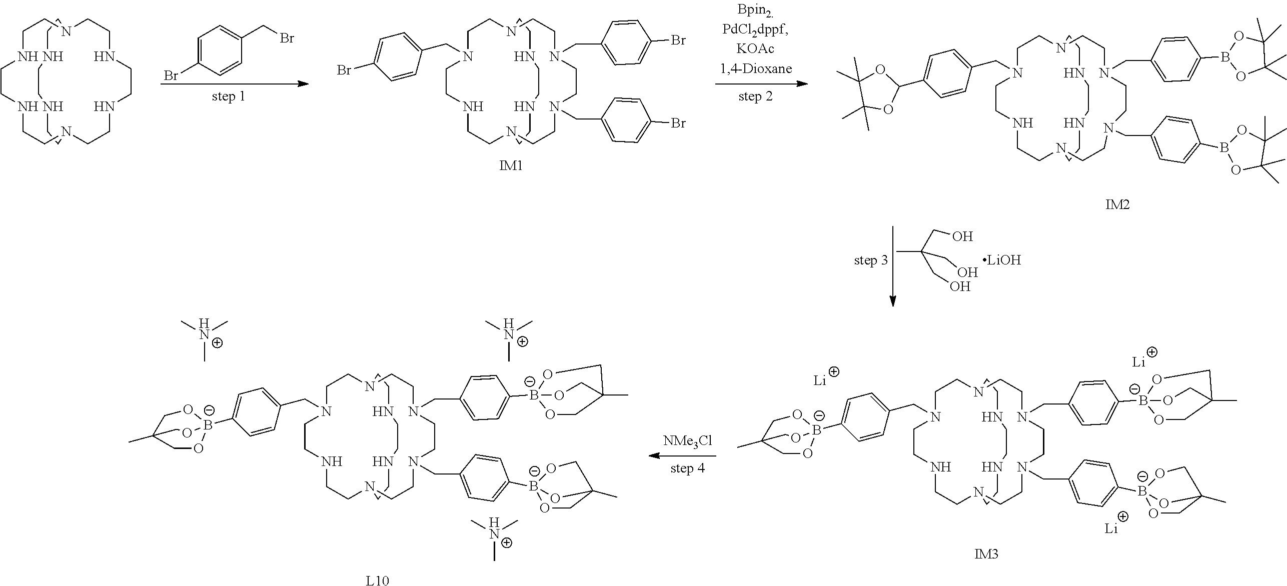

Step 1:4,7,13-tris(4-bromobenzyl)-1,4,7,10,13,16,21,24-octaazabicyclo [8.8.8] hexacosane (IM1): A solution of bromobenzylbromide (9.0 g, 36 mmol) in anhydrous THF (20 ml) was added dropwise to a stirring solution of L1 (3.7 g, 10 mmol) in 20 ml anhydrous THF at 0° C. The mixture was allowed to reach RT over a course of 1 h and stirring continued for an additional hour under reflux. The precipitated solid was filtered, washed with anhydrous THF and dried in vacuum. The powder was dissolved in a minimum amount of methanol (5-10 ml) and passed through a column of anion exchange resin (Amberlite IRA-410, 50 ml), which was previously converted into OH-form by treatment with methanolic solution of NaOH (20%, 20 ml) and washed neutral with methanol. The eluent was evaporated, yielding a crude product, which was purified by column chromatography (Al2O3 neutral, ethyl acetate: methanol 5:2). The solvents were removed in vacuum, yielding the title compound (3.8 g, 43%) as a mixture of constitutional isomers. This mixture was used for the next step without further separation.

Step 2:4,7,13-tris(4-(4,4,5,5-tetramethyl-1,3,2-dioxaborolan-2-yl)benzyl)-1,4,7,10,13,16,21,24-octaazabicyclo [8.8.8] hexacosane (IM2): A mixture of IM1 (3.8 g, 4.3 mmol), bis-(pinacolato)diboron (9.8 g, 38.7 mmol), potassium acetate (11.4 g, 116 mmol) and Pd(dppf)Cl2 (283 mg, 0.39 mmol) and anhydrous dioxane (100 ml) was heated at 90° C. for 24 h with stirring. The reaction mixture was cooled to room temperature and the volatiles were evaporated under reduced pressure. The residue was dissolved in water, extracted with dichloromethane (5×20 ml). The combined extracts were washed with brine, dried over anhydrous Na2SO4, filtered and evaporated to dryness. The crude product was purified by column chromatography on Al₂O₃ (neutral) yielding the title compound as a viscous, colorless oil (3.7 g, 84%)

1H NMR (300 MHZ, CDCl3): δ [ppm]=7.68 (d, 6H, Ar), 7.26 (d, 6H, Ar), 3.58 (m, 6H, CH2Ar), 2.33-2.76 (m, 36H), 1.20-1.35 (s, 36H); 11B NMR (300 MHz, CDCl3): δ [ppm]=33.9; MS (ESI): m/z=1019.8 (M+H)+

Step 3: Lithium 1,1′-(((7-(4-(4-methyl-2,6-dioxa-1-borabicyclo [2.2.1] heptan-1-uid-1-yl)benzyl)-1,4,7, 10, 13, 16,21,24-octaazabicyclo [8.8.8] hexacosane-4,16-diyl)bis(methylene))bis(4,1-phenylene))bis(4-methyl-2,6,7-trioxa-1-borabicyclo [2.2.2] octan-1-uide) (IM3): 1.7 mmol (1.0 eq.) of borolan, 5.1 mmol (3.0 eq.) of 1,1,1-tris(hydroxymethyl) ethane, 5.1 mmol (3.0 eq.) of LiOH and dry toluene (100 mL) were placed in a single neck round bottom flask. The mixture was boiled for 5 hours. The precipitate was filtered, washed with toluene and cyclohexane and dried in vacuum.

Yield: 72%; 1H NMR (300 MHZ, DMSO): δ [ppm]=7.65 (d, 6H, Ar), 7.21 (d, 6H, Ar), 3.52 (m, 6H, CH2Ar), 2.32-2.74 (m, 36H), 2.07-2.27 (m, 18H), 0.66 (s, 9H); MS (ESI): 1023.7 (A3-+2H+).

Step 4: Trimethylamine 1,1′-(((7-(4-(4-methyl-2,6-dioxa-1-borabicyclo [2.2.1] heptan-1-uid-1-yl)benzyl)-1,4,7,10, 13, 16,21,24-octaazabicyclo [8.8.8] hexacosane-4,16-diyl)bis(methylene))bis(4,1-phenylene))bis(4-methyl-2,6,7-trioxa-1-borabicyclo [2.2.2] octan-1-uide) (110): An excess (4.0 eq.) of an aqueous solution of trimethylamine hydrochloride was added dropwise to a stirred solution of IM3 (2.05 g, 2.0 mmol) in water (20 ml). A white precipitate formed upon addition. The reaction mixture was stored overnight at 0° C. in a fridge to complete precipitation of the product. The white solid was separated by filtration, washed with cold water (2×10 ml) and dried in vacuum at 60° C. In order to remove residual traces of water, the product was dissolved in THF and the solution was dried over molecular sieves (4 A) overnight before it was used for the synthesis of Ce-emitter.

### Synthesis of 2,2′,2″-((1,4,7,10,13,16,21,24-octaazabicyclo [8.8.8] hexacosane-4,7,13-triyl)tris(methylene))triphenol (I13)

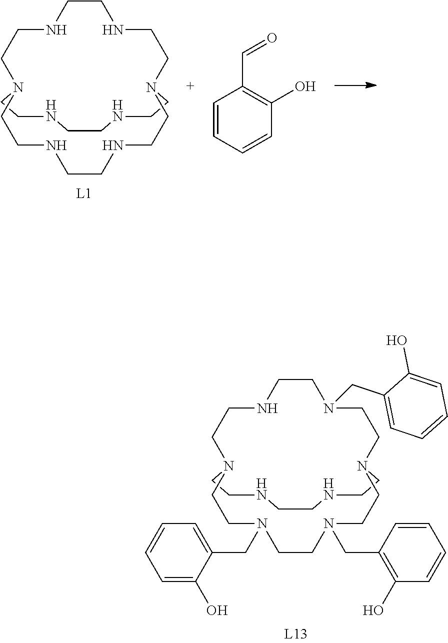

The reaction was carried out with standard Schlenk-technique. 5.4 mmol (1.0 eq.) of octaamine (I1) was dissolved in 35 ml of 1,2-dichloroethane. To this mixture was slowly added a solution of 16.2 mmol (3.0 eq.) of benzaldehyde in 35 ml of 1,2-dichloroethane. The mixture was treated with 21.6 mmol (4.0 eq.) of sodium triacetoxyborohydride followed by the addition of 21.6 mmol (4.0 eq.) of glacial acetic acid. The mixture was stirred at RT under Ar overnight. It was then quenched by the addition of dry NaHCO₃powder, and the product was extracted with chloroform. The solvent was removed under reduced pressure. The crude product was purified by flash chromatography.

Yield: 52%; 1H NMR (300 MHZ, CD3OD): δ [ppm]=7.35 (d, 3H, Ar), 7.17 (t, 3H, Ar), 6.90 (m, 6H, Ar), 3.39 (m, 6H, CH2Ar), 2.37-3.25 (m, 36H); MS (ESI): 689.5 (M+H)+.

### General Procedure of Synthesis of Bridged Aza-Crown Ligand Synthesis by Template-Reaction

Ligands 15, 16 and 17 were prepared by the following general procedure, which is a modification of a method described earlier: A. Brown, T. Bunchuay, C. G. Crane, N. G. White, A. L. Thompson, P. D. Beer, Chem. Eur. J. 2018, 24, 10434-10442.

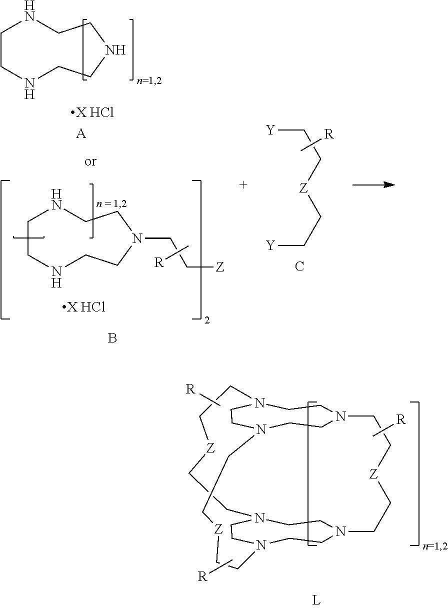

A suspension of 4.0 mmol (2.0 eq.) poly-aza-cycloalkane A or 2.0 mmol (1.0 eq.) of bis-poly-aza-cycloalkane B in 250 ml acetonitrile was treated with 6.0 mmol (3.0 eq.) or 8.0 mmol (4.0 eq.) Dihalide C. The mixture was stirred vigorously while 40 mmol (20 eq.) solid Na2CO3 were added in portions. The reaction was heated to reflux and stirring continued for 3 to 5 days. The hot mixture was filtered and the solution concentrated in vacuum. The residue was diluted with CH2Cl2 (150 ml) and again filtered and concentrated. The obtained solid was purified by flash-chromatography on aluminum oxid (neutral, Brockmann-I) using a CH2Cl2/MeOH 0-8% gradient. The obtained ligands were adsorbed on a column loaded with AmberChrom 50Wx2 (H+-form) cation exchange resin. The column was washed thoroughly with water and 1 M HCl. The ligand was eluted with 4 M HCl and concentrated in vacuum to dryness. The residue was dissolved in a small amount of water, adsorbed on a column loaded with Amberlite IRA-410 (OH—-form) anion exchange resin. The column was washed thoroughly with water and the ligand eluated with methanol. Concentration of the eluate yielded the Ligands 15, 16 and 17 as free base.

TABLE 3
Bridged crown ether ligands prepared using template reactions:

| Product | Code | Starting materials | |
|---|---|---|---|
| 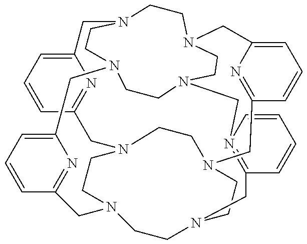 | I5 | 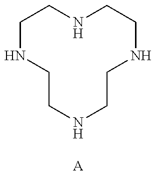 | 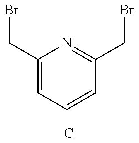 |
| 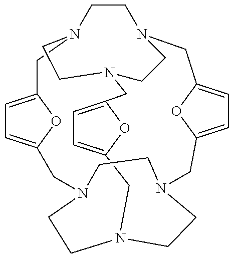 [SMILES: c1cc2oc1CN1CCN3CCN(CC1)Cc1ccc(o1)CN1CCN(CCN(CC1)Cc1ccc(o1)C3)C2] | I6 | 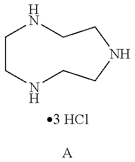 | 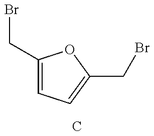 |
| 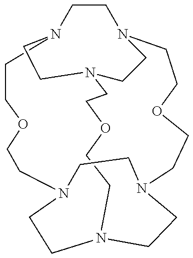 [SMILES: C1CN2CCN3CCOCCN4CCN(CCO1)CCN(CCOCCN(CC2)CC3)CC4] | I7 | 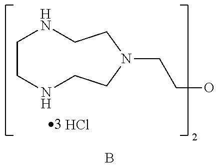 | 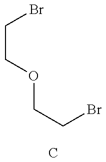 |

### General Procedure for the Preparation of Bis-Poly-Aza-Cycloalkanes

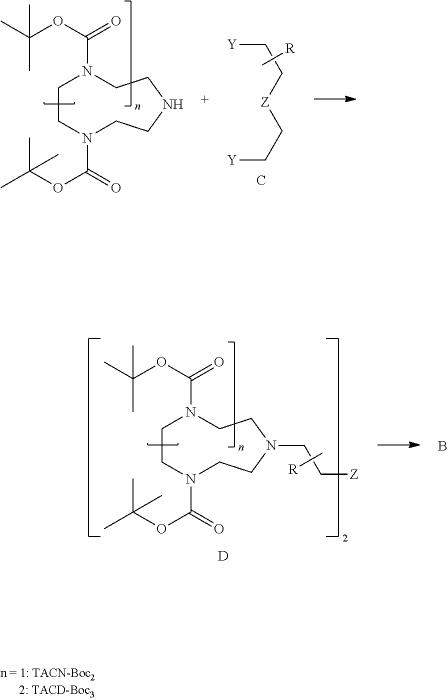

5.0 mmol (2.5 eq.) of TACN-Boc2 or TACD-Boc3 were dissolved in 100 ml acetonitrile. 10 mmol (5.0 eq.) potassium carbonate and 2.0 mmol (1.0 eq.) Dihalide C were added and the reaction heated to reflux for 2 days. The mixture was filtered and the solution concentrated in vacuum. The residue was purified by flash-chromatography on silica using a CH2Cl2/MeOH mixture to give the linked Bis(aza-cycloalkanes) D as oils.

2.0 mmol (1.0 eq.) of D were dissolved in 5 ml MeOH and treated with HCl/MeOH solution. The solution was stirred at room temperature overnight. The precipitate was filtered, washed with cold ether (3×20 ml) and dried in vacuum to give the bis-poly-aza-cycloalkanes D as their hydrochloride salts

### Synthesis of Ce3+ Emitters

P1. From Ce halogenides: Solution of cerium (III) halogenide (1 eq. 0.5 mmol) in anhydrous THF (10 ml) was added to a stirring solution of corresponding ligand (1 eq. 0.5 mmol) in THF or THF/dichloromethane (1:1 vol.) (10 ml). Precipitated product is separated by filtration, washed with THF, hexane and dried in vacuum

P2. From B1 compound and H-Ligands: This procedure was used, if starting ligand comprises moderately acidic protons (alcohols, azoles or trimethylamine salts of strong acid)

A solution of b1 (0,193 g, 0,277 mmol, 1,0 Eq.) in 5 ml of anhydrous THF was added dropwise to the solution of a ligand in 5 ml of anhydrous THF. All volatiles were removed in vacuum, residue was triturated with hexane, product is separated by filtration, washed with hexane and dried in vacuum. The crude products were sufficiently pure for most application. Analytically pure samples were obtained by recrystallization from an appropriate solvent or solvent mixtures.

P3. From b1 compound and H-form of anions: This procedure was applied to prepare cerium emitters from neutral ligands and H-form (preferably trimethylamine salt) of required anions.

To a solution of a ligand (1 eq.) and acidic form of corresponding anions (3 eq) in anhydrous THF, the solution of b1 (1 eq.) was added dropwise at RT. Stirring was continued at RT for 1 h, then all volatiles were removed in vacuum, residue was triturated by toluene. The solid product is separated by suction filtration, washed with toluene, hexane and dried in vacuum. The obtained crude product was sufficiently pure for most application. Analytically pure samples were obtained by recrystallization

P4 Gas phase synthesis of e10: This procedure was applied to prepare cerium emitters in a gas phase by co-evaporation of b1 as a cerium source and ligand comprises moderately acidic protons.

A sublimation tube was loaded with two sources, charged with ligand L2 (0.2 mmol (1.0 eq.) and Cerium compound b1 (0.2 mmol, 1.0 eq.). The tube was evacuated to 1.0E-4 hPa and heated to 170° C. using tubular glass oven (Büchi, B-585). Both materials simultaneously sublime and subsequently condense in the cold zone of sublimation tube thereby forming a thin film of corresponding emitter, as judged by UV-VIS/fluorescent measurements. The successful solvent free formation of the emitter was further confirmed by ESI-MS spectra.

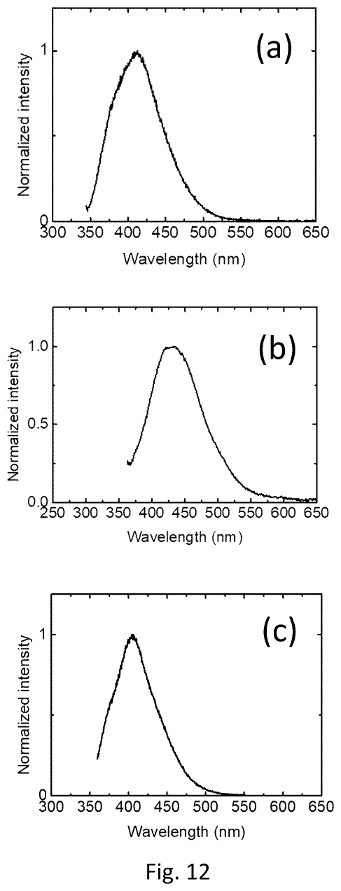

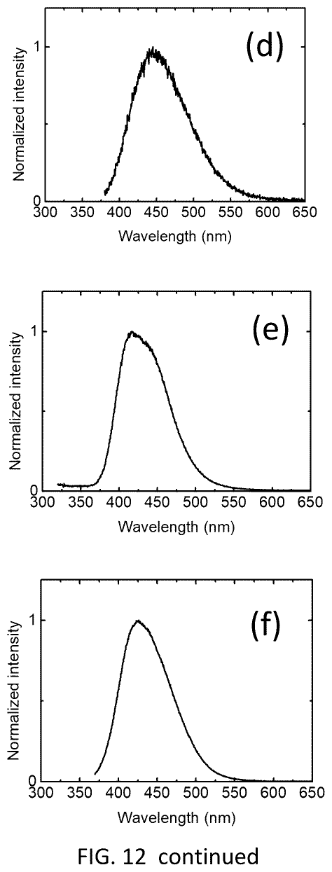

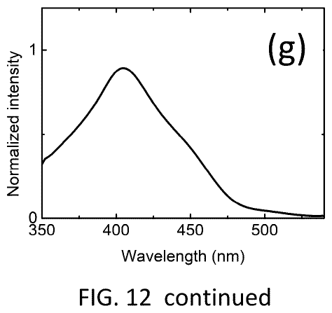

TABLE 5
Cerium Emitter compounds synthesized using method P1 to P4.

| | | | Elementar | Emission | | |
|---|---|---|---|---|---|---|
| | Yield | | analysis | max, nm | | |
| | (%)/ | | (calculated/ | (solvent)/ | PLQY, | LT, |
| Code | method | MS | found) | spectra | %* | ns** |
| e12 | 95/P1 | 170[M − 3I]3+ | | 435 (Methanol) | 46.6 | 127 |
| | | 318.6[(M − 2I)]2+ | | FIG. 12 (b) | | |
| e40 | 87/P1 | 170[M − 3I]3+ | | | | |
| e36 | 95/P2 | | | | | |
| e41 | 93/P2 | | | | | |
| e10 | 91/P2 | 1048.6 | Calc.: C, 34.39; | 406 (THF) | 72 | 76 |
| | | (M + H)+ | H, 4.33; N, 10.69. | FIG. 12 (c) | | |
| | | | Found.: C, 35.02; | | | |
| | | | H, 4.07; N, 10.86 | | | |
| e42 | 91/P2 | | | | | |
| e11 | 90/P2 | 748.4 | Calc.: C, 48.18; | 442 (Toluene) | 4.5 | 46 |
| | | (M + H)+ | H, 6.87; N, 26.22. | FIG. 12 (d) | | |
| | | | Found.: C, 48.52; | | | |
| | | | H, 6.71; N, 26.46. | | | |
| e33 | 84/P2 | 676.3 | Calc.: C, 47.98; | | | |
| | | (M + H)+. | H, 7.61; N, 16.58. | | | |
| | | | Found.: C, 48.02; | | | |
| | | | H, 7.83; N, 16.42. | | | |
| e43 | 62/P2 | 826.4 | Calc.: C, 56.71; | 418 (THF) | 4 | 40 |
| | | (M + H)+ | H, 6.96; N, 13.57. | FIG. 12 (e) | | |
| | | | Found.: C, 57.05; | | | |
| | | | H, 7.43; N, 13.78. | | | |
| e16 | /P1 | | | | | |
| e21 | /P1 | | | | | |
| e38 | /P1 | | | | | |
| e39 | /P3 | | | | | |
| e29 | 91/P3 | | | 424 (THF) | 82 | 95 |
| | | | | FIG. 12 (f) | | |
| e25 | /P3 | | | 407 (Methanol) | <1 | — |
| | | | | FIG. 12 (g) | | |
| e37 | /P2 | 1162.57 | Calc.: C, 55.82; | | | |
| | | (M + H)+ | H, 7.29; N, 9.64 | | | |
| | | | Found.: C, 56.15; | | | |
| | | | H, 7.73; N, 10.04. | | | |

*PLQY: Photo-Luminescence-Quantum-Yield measured in solution.
**Exponential decay time after optical pulsed excitation

### In-Situ Solvent-Free Formation of e10

Precursor materials (I2 and b1) were placed inside a high-vacuum chamber with two separate ceramic crucibles. Previously a quartz substrate was inserted into the chamber, above both sources to serve a substrate. The chamber was then evacuated using a pre-pump reaching 10-2 mbar and subsequently a turbopump to around 10-7 mbar. Then, I2 and b1 were slowly heated to approximately 150 and 60° C., respectively, ensuring a (mass) evaporation rate ratio of 3:2, corresponding to 1:1 molar ratio. The evaporation rate was monitored by separate quartz-crystal-monitors (QCM) and was kept constant throughout the process, at 1 Å/s and 0.6 Å/s, respectively. When the two powders started subliming, a sharp increase in chamber pressure was observed, reaching values of up to 5*10-5 mbar.

The co-evaporation of the two materials resulted in a film formation on the quartz substrate, of approximately 500 nm thickness. The film was completely transparent by eye. However, when excited with UV light at 340 nm, the film emitted a deep-blue color. The resulting emission spectrum was recorded and is displayed in FIG. 12 (a).

The emission spectrum recorded from the solid-state sample produced by co-evaporation of I2 and b1 overlaps perfectly with those emission spectra of e10 recorded in THF made by solution based synthesis methods P2 and P4, compare FIG. 12 (c). Thus, it was concluded that the correct emitter was formed in-situ by evaporating the precursor components separately.

### Methods for Layer Formation and Electroluminescent Devices (OLEDs)

A representative example of an organic electronic device and making thereoff according will now be described, including a detailed description of the fabrication process of the organic electronic device. Yet, it may be understood that neither the specific techniques for fabrication of the device, nor the specific device layout, nor the specific compounds are intended to limit the scope.

### MATERIAL DEFINITIONS

ITO: Indium tin oxide
PEDOT: PSS: Poly(3,4-ethylenedioxythiophene) polystyrene sulfonate
PVK: Poly(9-vinylcarbazole)
PPO21: 3-(Diphenylphosphoryl)-9-(4-(diphenylphosphoryl)phenyl)-9H-carbazole
SPPO1 9,9-Spirobifluoren-2-yl-diphenyl-phosphine oxide
BPhen: 4,7-Diphenyl-1,10-phenanthroline
Cs: Cesium
Al: Aluminium

PEDOT: PSS was purchased from Ossila with (1.3 to 1.7 wt. % (in water)). PVK was purchased from Sigma-Aldrich with an average molecular weight of 25,000-50,000. PPO21, Bphen, SPPO1 were purchased from Lumtec and were used as-received.

### Coevaporation of Host and Emitter

Pre-mixed SPPO1 and emitter e10 were co-sublimed from a single source to form a mixed layer. Both substances were dissolved in dichloromethane with subsequent solvent evaporation to give a 35 wt % mixture of e10 in SPPO1 as a powder. This powder (70 mg) was placed in the tubular sublimation apparatus equipped with an oven (Buchi). After reaching a vacuum of 1.0E-4 hPa, a gradual rise in temperature up to 170° C. was started. Up to this temperature, no material transfer to the cool side of the glass tube was observed. Material transfer was observed by rising the temperature further to 180° C. After operating the sublimation tool at this temperature for 8 h the tool was left to cool down. The condensed substance (about 70% of the originally present material, 30% remained in the heating source) was investigated by ESI-MS and hereby confirmed the substantial presence of both, SPPO1 and e10. No more e10 was left in the source residue, which—according to ESI-MS—only contained pure SPPO1.

In a sequential experiment, 6 mg of the sublimed powder containing a mixture of SPPO1 and e10 was used for re-sublimation. At the same conditions as above this material was now completely sublimed, i.e. the sublimation yield was within error 100%. Again, ESI-MS confirmed that the sublimed, condensed material was SPPO1 and e10.

### Device Fabrication

The organic electronic device was prepared with the following layer sequence:
ITO/PEDOT: PSS/PVK/PPO21: e29/BPhen/Cs/Al.

The organic electronic device was fabricated on a 1″×1″ size glass substrate, pre-coated with a transparent ITO anode. The substrate was cleaned with various solvents in an ultrasonic bath and subsequently exposed to UV oxygen plasma for 10 min. PEDOT: PSS was filtered and treated in an ultrasonic bath for 30 min. 200 μl of the solution was spin-coated on the substrate at 2400 rpm for 60 s, resulting in about 30 nm thick film. The coated substrate was annealed at 120° C. for 30 mins to remove residual solvents. Subsequently, the coated substrate was transferred to a nitrogen atmosphere glovebox, where an electron blocking layer with high triplet energy, namely the hole transporting polymer PVK, was spin-coated from degassed chlorobenzene, 200 μl, 10 mg/ml, at 4000 rpm for 60 s, resulting in about 20 nm thick film. Afterwards the coated substrate was annealed at 150° C. for 10 mins to remove residual solvents. Subsequently, the emissive layer containing PPO21: e29 was deposited on the substrate. PPO21 and e29 were individually dissolved in degassed dichloroethane at 10 mg/ml respectively. The solutions were mixed at 10:1 ratio and 200 μl of the mixture was spin-coated on the substrate at 6000 rpm for 60 s, resulting in about 15 nm thick film, which was further processed without annealing.

Next, the substrate was transferred in a sealed transferring container to a thermal evaporation chamber operating at around 10-7 mbar pressure. Here, a 40 nm film of BPhen was thermally evaporated, followed by a thin Cs interlayer. The device was finished by evaporating a 100 nm film of cathode Al. The Al deposition was carried out using a structured shadow mask, such that the resulting overlap of pre-structured ITO, organic layers and Al gave an active area of 6.25 mm2. Subsequently, the device was transferred to a nitrogen atmosphere glovebox and encapsulated by gluing an additional encapsulating glass substrate on top to prevent oxygen- and water-induced degradation. The electrical and electroluminescent properties of the device were measured using a Keithley 2400 source meter and by placing the device in an Ulbricht sphere with a calibrated Si photodiode and OceanOptics spectrometer.

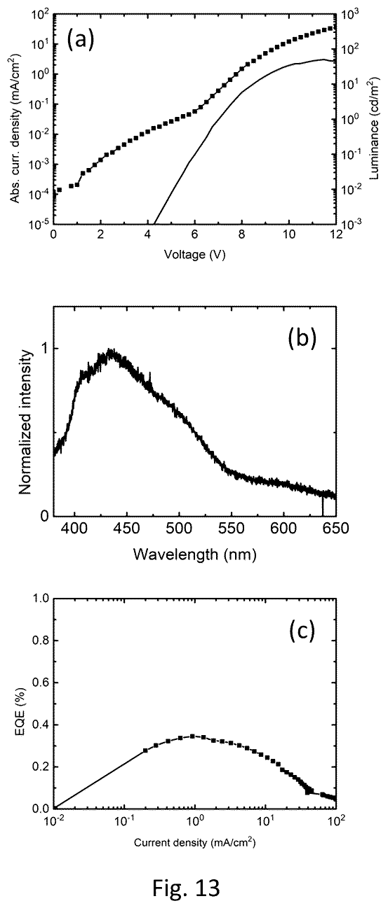

The device showed turn-on at 4 V, the current density reached 1 mA/cm² at 8 V and 10 mA/cm² at 10 V, which may represent the typical operating conditions for an organic electronic device used in flat-panel displays. Resulting current-voltage-luminance, electroluminescence spectrum and EQE curves are presented in FIG. 13.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A metal-organic coordination compound, wherein the coordination compound comprises at least one Cerium (III) coordinated by a poly macrocyclic organic ligand having the formula 1-1 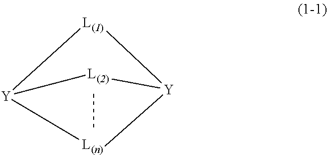
wherein n is an integer of at least 3,
Y at each occurrence is independently a n-valent group containing 1 to 30 atoms,
L at each of its n occurrences is independently a divalent organic group, wherein L has a sequence of at least 3 atoms connecting both Y in case that Y is a cyclic group connected with each L via different ring atoms of Y,
wherein L has a sequence of at least 5 atoms connecting both Y in case that Y is connected with each L via the same atom of Y,
wherein the poly macrocyclic organic ligand comprises at least three nitrogen atoms and is one- to threefold negatively charged.

2. The coordination compound according to claim 1, wherein
each Y is independently selected from B, B⁻-R, N, P, C-R, or
each Y is independently selected from 3- to 15-membered cyclic organic groups that can contain one to four hetero atoms selected from N, B, P, O, S, and is connected with each L via non-neighboring carbon or hetero atoms,
wherein R is hydrogen or any covalently bound substituent being identical or different in each occurrence.

3. The coordination compound according to claim 1, wherein the poly macrocyclic organic ligand is either non-aromatic (aliphatic) or comprises aromatic building blocks containing at most 8 aromatic carbon or hetero atoms, whereby various aromatic building blocks are being separated from each other by at least one aliphatic carbon or hetero atom.

4. The coordination compound according to claim 1, wherein each Y is a cyclic organic group having a sequence of ring atoms composed of sub-sequences-N—C—C— or —O—C—C—.

5. The coordination compound according to claim 1, wherein each Y is a macrocyclic organic group comprising 9 to 12 ring atoms containing 3 or 4 hetero atoms selected from N or O.

6. The coordination compound according to claim 1, wherein each Y is N.

7. The coordination compound according to claim 1, wherein each L has a sequence of atoms connecting both Y composed of sub-sequences —N—C—C— or —O—C—C—.

8. The coordination compound according to claim 1, wherein the poly macrocyclic organic ligand is threefold negatively charged and in combination with a trivalent Cerium cation forms a complex of neutral charge.

9. The coordination compound according to claim 1, wherein the coordination compound comprises at least one anion that is not covalently linked to the poly macrocyclic organic ligand.

10. The coordination compound according to claim 9, wherein the at least one anion that is not covalently linked to the poly macrocyclic organic ligand comprises more than atoms or has a mass of more than 128 g/mol or comprises more than atoms and has a mass of more than 128 g/mol.

11. The coordination compound according to claim 9, wherein the singly negatively charged anion comprises boron.

12. The coordination compound according to claim 1, wherein the poly macrocyclic organic ligand is a bis cyclic cryptand.

13. The coordination compound according to claim 1, with the poly macrocyclic organic ligand having a structure according to formula 1a-1g: 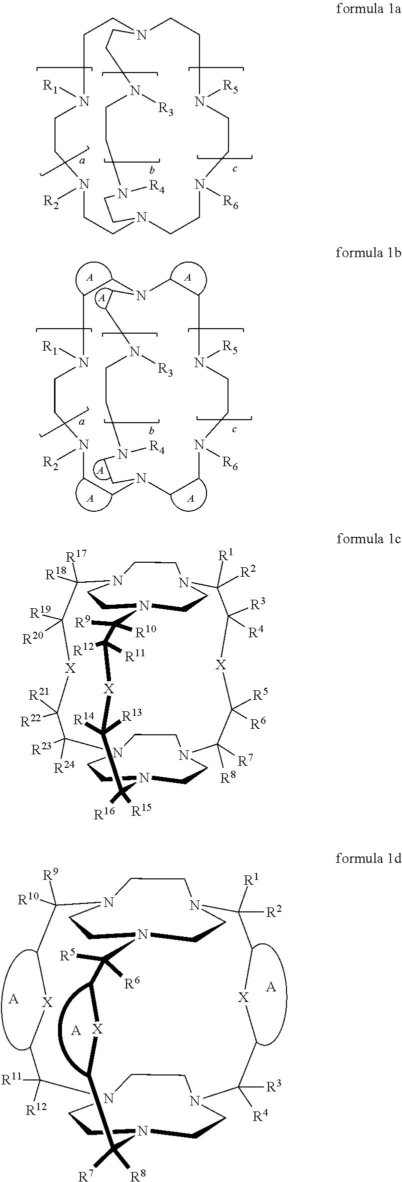 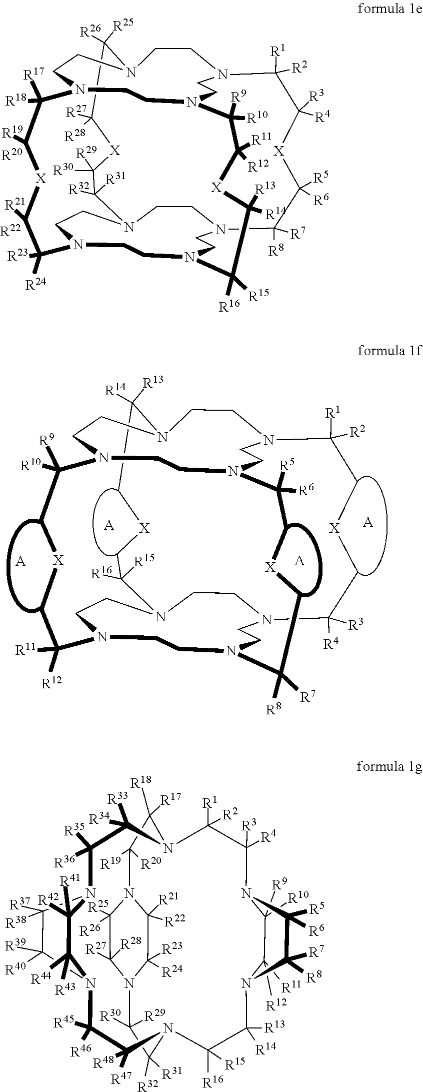
wherein
R₁-R₄₈ independently in each occurrence represent hydrogen, deuterium, Cl, F, Br, CN, NO₂ or an organyl group,
a, b, c in formula 1a and 1b are each independently an integer of 0 or more, 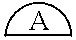
In formula 1b, 1d, 1f independently in each occurrence represents a divalent cyclic organic group, wherein one or more of the ring-forming carbon atoms can be substituted or can be part of a cyclic group,
X in formula 1c, and 1e is independently in each occurrence selected from
B-R,N-R,P-R,C-R-R,O,S
X in formula 1d, and 1f is independently in each occurrence selected from
B,B-R,N,N-R,P,P-R,C-R,C-R-R,O,S
wherein R is hydrogen or deuterium or any covalently bound substituent being identical or different in each occurrence.

14. The coordination compound according to claim 1, wherein L has a sequence of at least 5 atoms.

15. The coordination compound according to claim 1, wherein each Y is independently selected from 6- to 12-membered cyclic organic groups that can contain one to four hetero atoms selected from N, B, P, O, S, and is connected with each L via non-neighboring carbon or hetero atoms.

16. The coordination compound according to claim 1, wherein the poly macrocyclic organic ligand comprises monoanionic groups, with the monoanionic groups having the structure —CH₂—CRR—X with X being a monoanionic atom selected from O and S, or the monoanionic group has the structure 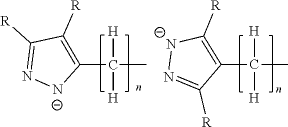
with n being an integer equal to one or more and with each R independently being H, substituted or unsubstituted C₁₋₁₂-alkyl, substituted or unsubstituted aryl, halogen, and wherein two groups R can be covalently linked to form a cyclic group.

17. The coordination compound according to claim 16, with the coordination compound having the structure 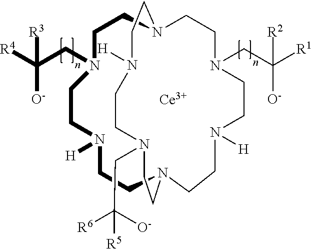
with n being an integer equal to one or more and with R₁-R₆ independently being H, deuterium, substituted or unsubstituted C₁₋₁₂-alkyl, substituted or unsubstituted aryl, halogen, and wherein two groups R can be covalently linked to form a cyclic group.

18. A mixture comprising
a second electrically neutral organic compound, and
a metal-organic coordination compound, wherein the coordination compound comprises at least one Cerium (III) coordinated by a poly macrocyclic organic ligand having the formula 1-1 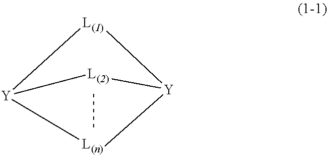
wherein n is an integer of at least 3,
Y at each occurrence is independently a n-valent group containing 1 to 30 atoms,
L at each of its n occurrences is independently a divalent organic group, wherein L has a sequence of at least 3 atoms connecting both Y in case that Y is a cyclic group connected with each L via different ring atoms of Y,
wherein L has a sequence of at least 5 atoms connecting both Y in case that Y is connected with each L via the same atom of Y,
wherein the poly macrocyclic organic ligand comprises at least three nitrogen atoms and can be as defined in claim 1,
wherein the coordination compound is imbedded into the at least one second electrically neutral organic compound,
wherein the second organic compound has a triplet energy higher than 2.5 eV, or wherein the coordination compound has a higher hole affinity compared to the second electrically neutral organic compound, or wherein the second organic compound has a triplet energy higher than 2.5 eV and the coordination compound has a higher hole affinity compared to the second electrically neutral organic compound,

19. The mixture according to claim 18, wherein L has a sequence of at least 5 atoms.

20. The mixture according to claim 18, wherein the second organic compound has a triplet energy higher than 2.7 eV.

21. A polymeric compound comprising a metal-organic coordination compound, wherein the coordination compound comprises at least one Cerium (III) coordinated by a poly macrocyclic organic ligand having the formula 1-1 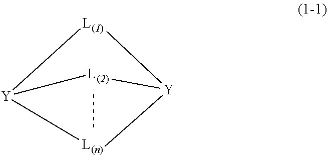
wherein n is an integer of at least 3,
Y at each occurrence is independently a n-valent group containing 1 to 30 atoms,
L at each of its n occurrences is independently a divalent organic group, wherein L has a sequence of at least 3 atoms connecting both Y in case that Y is a cyclic group connected with each L via different ring atoms of Y,
wherein L has a sequence of at least 5 atoms connecting both Y in case that Y is connected with each L via the same atom of Y,
wherein the poly macrocyclic organic ligand comprises at least three nitrogen atoms and can be as defined in claim 1, and
wherein a polymer with a molecular weight Mn above 1000 g/mol, wherein the coordination compound is covalently attached to the polymer backbone.

22. The polymeric compound according to claim 21, wherein L has a sequence of at least 5 atoms.

23. An organic electronic device, comprising:
a first electrode;
a second electrode; and
an organic layer arranged such that it is electrically interposed between the first and second electrodes,
wherein the organic layer comprises a metal-organic coordination compound,
wherein the coordination compound comprises at least one Cerium (III) coordinated by a poly macrocyclic organic ligand having the formula 1-1 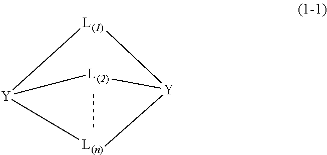
wherein n is an integer of at least 3,
Y at each occurrence is independently a n-valent group containing 1 to 30 atoms,
L at each of its n occurrences is independently a divalent organic group, wherein L has a sequence of at least 3 atoms connecting both Y in case that Y is a cyclic group connected with each L via different ring atoms of Y,
wherein L has a sequence of at least 5 atoms connecting both Y in case that Y is connected with each L via the same atom of Y,
wherein the poly macrocyclic organic ligand comprises at least three nitrogen atoms and is as defined in claim 1.

24. An organic electronic device of claim 23, wherein L has a sequence of at least 5 atoms.

25. The organic electronic device of claim 23, wherein the organic electronic device is an optoelectronic device, the optoelectronic device being at least one of an organic light emitting diode, an organic photodetector, or a photovoltaic cell.

26. A method of forming an organic device, the method comprising:
forming a layer of the coordination compound according to claim 1;
depositing the layer from a gas phase, in particular using an evaporation or sublimation or carrier gas process, or by a solution-based process.

27. The method according to claim 26, wherein forming the layer comprises forming a first layer comprising the organic poly macrocyclic ligand and forming a second layer directly in contact with the first layer, wherein the second layer comprises a Cerium salt.

28. The method according to claim 26, wherein forming the layer comprises evaporation or sublimation of the organic poly macrocyclic ligand and evaporation or sublimation of a Cerium salt whereby the evaporation or sublimation proceeds sequentially or simultaneously.

29. A method of forming an organic device according to claim 26, wherein the coordination compound is transferred into gas phase from a blend of the coordination compound with at least one second material, with the second material is present in the blend between 1 and 99 vol %.
